SEQUENCE LISTING (5 sequences)

SEQ ID NO: 1 - DNA, 17 bp, Artificial Sequence
  source: /note=“Description of Artificial Sequence Synthetic primer”
gctcgactatgtcaacg

SEQ ID NO: 2 - DNA, 19 bp, Artificial Sequence
  source: /note=“Description of Artificial Sequence Synthetic primer”
ccaagagaatgttctctgg

SEQ ID NO: 3 - DNA, 17 bp, Artificial Sequence
  source: /note=“Description of Artificial Sequence Synthetic primer”
ccagatcactcctgagc

SEQ ID NO: 4 - DNA, 21 bp, Artificial Sequence
  source: /note=“Description of Artificial Sequence Synthetic primer”
ccagatatcaactttcggacc

SEQ ID NO: 5 - protein, 7 aa, Artificial Sequence
  source: /note=“Description of Artificial Sequence Synthetic peptide”
RRRSLLE

(19) United States
(12) Patent Application Publication
(10) Pub. No.: US 2012/0208204 A1
(43) Pub. Date: Aug. 16, 2012

(54) Compositions and Methods for Inhibiting Tumor Growth

(75) Inventors: Amy Baldwin, Douglasville, GA (US); Dorre Grueneberg, Newton, MA (US); Ed Harlow, Boston, MA (US); Jun Xian, Sharon, MA (US); Karl Munger, Newton, MA (US); Karin Hellner, Oxford (GB); Marcie Glicksman, Winchester, MA (US); Ross Stein, Kansas City, MO (US); Gregory Cuny, Cambridge, MA (US)

(73) Assignees: The Brigham and Women's Hospital, Inc., Boston, MA (US); Peresident and Fellows of Harvard College, Cambridge, MA (US)

(21) Appl. No.: 13/376,322

(22) PCT Filed: Jun. 3, 2010
(86) PCT No.: PCT/US10/37280

§ 371 Date: Apr. 19, 2012

Related U.S. Application Data
(60) Provisional application No. 61/183,851, filed on Jun. 3, 2009.

(51) Int. Cl. G01N 21/64; C12N 5/09; C12Q 1/48; C12N 5/071

(52) U.S. Cl. 435/7.1; 435/375; 435/15

(57) ABSTRACT

The invention provides methods and compositions for inhibiting p53-inactivated cancers. Cancer cells are preferentially inhibited compared to normal cells by inhibiting tumor survival kinases that are required for growth of tumor cells but not normal cells.

### FIELD OF THE INVENTION

This invention relates to compounds and methods for cancer therapy.

### BACKGROUND OF THE INVENTION

The role of p53 as a tumor suppressor is generally attributed to its ability to stop the proliferation of precancerous cells by inducing cell-cycle arrest or apoptosis. This tumor suppressor gene is mutated in many human cancers and results in the loss of a cell's ability to survey for DNA damage. Inactivation or disruption of the p53 tumor suppressor gene is a common event in the development of most types (50-80%) of human cancers.

### SUMMARY OF THE INVENTION

The present invention provides compounds and methods to preferentially or specifically target tumor cells, e.g., inhibiting their proliferation or decreasing their survival, while sparing normal cells. Non-tumor cells are spared, because the compounds inhibit a kinase that becomes necessary for survival only when the process of carcinogenesis is initiated and remains necessary after the cell becomes cancerous. Identification of such specific therapeutic agents was possible only after elucidating a family of kinases that are not needed in normal cells but are necessary for survival of tumor cells. Such kinases are characterized or classified as tumor survival kinases. These kinases become essential in cells in which p53 is deficient, e.g., mutated, inactivated, or otherwise compromised or reduced. The compounds are used to inhibit proliferation or kill p53-deficient tumors in individuals, e.g., human patients, that have been diagnosed with a p53-deficient tumor.

Inhibitors of these kinases are superior to many existing anti-tumor drugs, because they preferentially act on p53-deficient tumor cells compared to non-tumor cells or cells in which p53 expression or activity is normal. Tumor survival kinases include serum- and glucocorticoid-induced protein kinase (SGK) and p21-activated kinase (PAK). For example, a method of inhibiting proliferation or decreasing proliferation of a p53-deficient tumor cell involves contacting the tumor cell with a composition comprising an inhibitor of SGK2, PAK3, or CDK7. The compounds inhibit proliferation of tumor cells or precancerous cells.

In one embodiment, the compounds inhibit the enzymatic activity or expression of a p53-dependent tumor cell survival kinase, e.g., PAK3 or SGK2, thereby reducing cell proliferation and/or causing death of the tumor cell. p53-deficient cells are contacted with an inhibitor of a tumor survival kinase. The p53-deficient cell is a p53 deficient tumor cell, a human papilloma virus (HPV)-infected cell (e.g., a non-tumor cell), or a non-tumor cell expressing an HPV oncoprotein. p53-deficient tumors affect many different tissue types. For example, the compounds are administered to a subject diagnosed as suffering from or at risk of developing a p53-deficient cell condition such as cancer or a precancerous lesion or mass. The cell to be treated is, e.g., a tumor cell or tumor cell line of a tissue type selected from the group consisting of breast, cervix, uterus, bladder, brain, lung, esophagus, liver, prostate, colon, brain (e.g., glioblastoma). Mutations associated with p53 deficiency (decrease or absence of expression level or enzymatic activity) is also associated a variety of sarcomas and leukemias.

In one example, the method involves contacting the cell, e.g., a tumor cell, with a composition comprising an inhibitor of PAK3, wherein said inhibitor comprises the structure of PAK3 inhibitor Chemotype 4

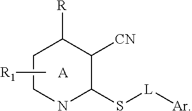

Inhibitors that belong to this chemotype group include LDN-0211958, LDN-0211959, LDN-0026056, LDN-0211955, LDN-0041012, and LDN-0028618. In another example, cells are contacted with a composition comprising an inhibitor of PAK3, wherein said inhibitor comprises the structure of PAK3 inhibitor Chemotype 8

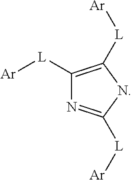

Exemplary compounds include LDN-0044878 or LDN-0091420.

In yet other example, the method of inhibiting proliferation of or killing a p53-deficient cell is carried out by contacting the cell with a composition comprising any one of the inhibitors shown in FIGS. 9A-T, e.g., inhibitor that have general structure selected from PAK3 inhibitor Chemotypes 1, 2, 3, 3a, 3b, 3c, 3d, 4, 5, 6, 7, 8, 8a, 9, 10, 11, 12, 13, 14. Other PAK3 inhibitory compounds useful in the these methods include LDN-0047862, -0009460, -0042112, -0097519, -0096422, -0111371, -0086947, -001731, -0080086, and -0097728.

The invention also includes methods of inhibiting proliferation of or killing a p53-deficient cell by targeting tumor survival kinase, SGK2. This method involves contacting the cell, e.g., the cell types described above, with a composition comprising an inhibitor of SGK2 that comprises the structure of SGK inhibitor chemotype 1A

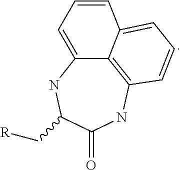

An exemplary composition comprises LDN-0149188. In another example, the method is carried out using an inhibitor of SGK2, wherein said inhibitor comprises the structure of SGK inhibitor chemotype 2A

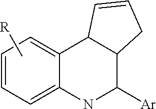

such as LDN-0144705 or LDN-0144676. In yet another example, the method involves contacting cells with a composition comprising an inhibitor of SGK2, wherein said inhibitor comprises the structure of SGK inhibitor chemotype 4

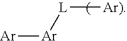

An exemplary compound that belongs to SGK inhibitor group chemotype 4 is LDN-0169731. Other useful SGK inhibitors comprise a general structure selected from SGK2 inhibitor Chemotypes 1, 1A, 1B, 2, 2A, and 4 as exemplified by compounds shown in FIGS. 11-13 as well as those shown in FIG. 14 (e.g., LDN-0181476 or LDN-0187289).

Analogues or derivatives of the aforementioned compounds are also useful in the described methods provided that the structure or chemical formulas comply with the general structures shown in FIG. 10 (for PAK3 inhibitor derivatives) or FIG. 15 (for SGK2 inhibitor derivatives).

A method of identifying a tumor survival kinase comprises synthetically inhibiting expression of a tumor-associated gene and expression of at least one candidate kinase gene. A decrease in tumor cell survival in the presence of inhibition of both genes compared to the level of tumor cell survival in the presence of inhibition of solely the tumor-associated gene (e.g., p53) indicates that the candidate kinase gene is a tumor survival kinase. For example, kinase targets are identified by depleting p53 (or other kinases) by infection with a lentiviral shRNA. Tumor survival kinases are identified by detecting combinations that lead to pronounced decreased in cell viability. For example, co-depletion of p53 and PAK3 or SGK2 resulted in a dramatic decrease in cell proliferation/viability, whereas depletion of an unrelated kinase, MAP3K8, lead to a similar effects in control and p53 depleted cells. This synthetic lethality approach is useful to identify tumor survival kinases, which are useful targets for anti-tumor drugs.

A method of identifying an anti-tumor agent for inhibition of p53 deficient tumor cells, is carried out by contacting tumor survival kinase with a candidate compound and determining whether the candidate compound inhibits enzymatic activity of the kinase. A reduction in a level of activity in the presence of the candidate compound compared to that in the absence of the candidate compound indicates that the candidate compound preferentially inhibits p53 deficient tumor cells. A method of identifying an anti-tumor agent for inhibition of p53 deficient tumor cells is carried out by contacting a cell dependent upon a tumor survival kinase with a candidate compound and determining whether the candidate compound inhibits survival or proliferation of the cell. A reduction in a level of survival or proliferation in the presence of the candidate compound compared to that in the absence of the candidate compound indicates that the candidate compound preferentially inhibits p53 deficient tumor cells. Exemplary

tumor survival kinases include those described above—serum- and glucocorticoid-induced protein kinase (SGK), a p21-activated kinase (PAK), as well as a cyclin-dependent protein kinase (CDK) such as CDK7.

Compounds identified by such screens are useful for inhibiting proliferation of or killing a p53-deficient cells such as tumor cells. The compounds inhibit or decreases enzymatic activity of SGK2 or PAK3. A reduction in a level of the activity in the presence of the candidate compound compared to that in the absence of the candidate compound indicates that the candidate compound preferentially inhibits p53 deficient tumor cells. For example, enzymatic activity is reduced by 20%, 50%, 75%, or more (e.g., 2-fold, 5-fold, 10-fold, or more).

Preferably, the tumor survival kinases are SGK2 (SGK2 (GENBANK Accession No. NM_{—}170693), PAK3 (NM_{—}002578), and CDK7 (NM_{—}001799) GENBANK Accession No. NM_{—}016276.3, NP_{—}057360.2), PAK3 (NM_{—}002578.2, NP_{—}002569.1), or CDK7 (NM_{—}001799.3, NP_{—}001790.1).

A cell dependent upon a tumor survival kinase is a cancer cell, e.g., a p53 deficient tumor cell, or a human papilloma virus (HPV)-infected cell, or a non-tumor cell expressing an HPV oncoprotein. The tumor cell to be treated or tumor cell line to be tested is of a tissue type selected from the group consisting of bladder, brain, breast, cervix, colon, esophagus, head and neck, liver, lung, pancreas, prostate, soft tissue, stomach, uterus, leukemias and lymphomas.

In one embodiment, the compounds of the disclosure include a heterocyclic group comprising at least two nitrogen atoms. Examples of suitable diazaheterocycles include imidazolidine, pyrazolidine, piperazine, pyrimidine, pyridazine, pyrazine, and annulated bicyclic compounds comprising such diazaheterocycles. In one group of preferred embodiments, the compounds are diaza heterocyclic compounds that further comprises an amide moiety. The amide moiety may be part of a cyclic portion of the compound, and/or may be part of a linear portion of the compound.

The compounds to be used in the methods described herein are purified. For example, the compounds are chemically synthesized and separated from starting ingredients and by-products using known methods such as chromatographic techniques. A purified compound comprises at least 75%, 80%, 90% or 99%-100% by weight (w/w).

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

As used herein, the terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Reference to specific alkyl groups is meant to include all constitutional isomers that exist for that group. Generally, although again not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and the terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C₁-C₆ alkoxy” or “lower alkoxy” herein may, for example, may contain 1 to 3 carbon atoms, and as a further example, such substituents may contain 1 or 2 carbon atoms (i.e., methoxy and ethoxy).

The term “aryl” as used herein, and unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “aralkyl” refers to an alkyl group with an aryl substituent, and the term “alkaryl” refers to an aryl group with an alkyl substituent, wherein “alkyl” and “aryl” are as defined above. In general, aralkyl and alkaryl groups herein contain 6 to 30 carbon atoms. Aralkyl and alkaryl groups may, for example, contain 6 to 20 carbon atoms, and as a further example, such groups may contain 6 to 12 carbon atoms. The term “amino” is used herein to refer to the group —NZ¹Z² wherein Z¹ and Z² are hydrogen or nonhydrogen substituents, with nonhydrogen substituents including, for example, alkyl, aryl, alkenyl, aralkyl, and substituted and/or heteroatom-containing variants thereof. The terms “halo” and “halogen” are used in the conventional sense to refer to a chloro, bromo, fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

“Hydrocarbyl” refers to univalent hydrocarbyl radicals containing 1 to about 30 carbon atoms, including 1 to about 24 carbon atoms, further including 1 to about 18 carbon atoms, and further including about 1 to 12 carbon atoms, including linear, branched, cyclic, saturated and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

The term “cyclic” as used herein refers to a molecule, linkage, or substituent, that is or includes a circular connection or atoms. Unless otherwise indicated, the term “cyclic” includes aromatic, alicyclic, substituted, unsubstituted, heteroatom-containing moieties, and combinations thereof.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation: functional groups such as halo, oxo (═O), hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—C(═O)-alkyl) and C₆-C₂₀ arylcarbonyl (—C(═O)-aryl)), acyloxy (—O-acyl), C₂-C₂₄ alkoxycarbonyl (—C(═O)—O-alkyl), C₆-C₂₀ aryloxycarbonyl (—C(═O)—O-aryl), halocarbonyl (—C(═O)—X where X is halo), C₂-C₂₄ alkylcarbonato (—O—C(═O)—O-alkyl), C₆-C₂₀ arylcarbonato (—O—C(═O)—O-aryl), carboxy (—COOH), carboxylato (—COO⁻), carbamoyl (—C(═O)—NH₂), mono-substituted C₁-C₂₄ alkylcarbamoyl (—C(═O)—NH(C₁-C₂₄ alkyl)), di-substituted alkylcarbamoyl (—C(═O)—N(C₁-C₂₄ alkyl)₂), mono-substituted arylcarbamoyl (—C(═O)—NH-aryl), thiocarbamoyl (—C(═S)—NH₂), carbamido (—NH—C(═O)—NH₂), cyano (—CN), isocyano (—N⁺≡C⁻), cyanato (—O—C≡N), isocyanato (—O—N⁺≡C⁻), isothiocyanato (—S—C≡N), azido (—N═N⁺═N⁻), formyl (—C(═O)—H), thioformyl (—C(═S)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—C(═O)-alkyl), C₅-C₂₀ arylamido (—NH—C(═O)-aryl), imino (—CR═NH where R=hydrogen, C₁-C₂₄ alkyl, C₅-C₂₀ aryl, C₆-C₂₀ alkaryl, C₆-C₂₀ aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—S(O)-alkyl), C₅-C₂₀ arylsulfinyl (—S(O)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O⁻)₂), phosphinato (—P(O)(O⁻)), phospho (—PO₂), and phosphino (—PH₂), mono- and di-(C₁-C₂₄ alkyl)-substituted phosphino, mono- and di-(C₅-C₂₀ aryl)-substituted phosphino; and the hydrocarbyl moieties C₁-C₂₄ alkyl (including C₁-C₁₈ alkyl, further including C₁-C₁₂ alkyl, and further including C₁-C₆ alkyl), C₂-C₂₄ alkenyl (including C₂-C₁₈ alkenyl, further including C₂-C₁₂ alkenyl, and further including C₂-C₆ alkenyl), C₂-C₂₄ alkynyl (including C₂-C₁₈ alkynyl, further including C₂-C₁₂ alkynyl, and further including C₂-C₆ alkynyl), C₅-C₃₀ aryl (including C₅-C₂₀ aryl, and further including C₅-C₁₂ aryl), and C₆-C₃₀ aralkyl (including C₆-C₂₀ aralkyl, and further including C₆-C₁₂ aralkyl). In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated. Where appropriate and unless otherwise specified, the terms “substituted” and “substituent” when used in the context of cyclic groups such as aromatic and alicyclic groups are meant to include fused rings and other multiple ring systems. For example, a substituted aryl group includes such groups as naphthyl and anthracenyl.

When the term “substituted” appears prior to a list of possible substituted groups, it is intended that the term apply to every member of that group. For example, the phrase “substituted alkyl and aryl” is to be interpreted as “substituted alkyl and substituted aryl.” By two moieties being “connected” is intended to include instances wherein the two moieties are directly bonded to each other, as well as instances wherein a linker moiety (such as an alkylene or heteroatom) is present between the two moieties.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

The compounds and methods described herein have numerous advantages over existing treatments because they target tumor cells, e.g., tumor cells in which p53 expression is deficient or lost, and spares normal no-tumor cells or cells that are characterized by normal p53 expression.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following description of the preferred embodiments thereof, and from the claims. References cited, including the contents of GENBANK Accession Numbers are hereby incorporated by reference.

### BRIEF DESCRIPTION OF THE FIGURES

FIG. 1 is a list showing identification of protein kinases that become essential as a consequence of HPV oncoprotein expression in primary human keratinocytes. HeLa, SiHa and CaSki cervical carcinoma (CxCa), high passage (HP) and low-passage (LP) HPV16 immortalized keratinocytes as well as human foreskin keratinocytes (HFKs) engineered to express the entire HPV16 early coding region (ER) or E6 and/or E7 oncoproteins were infected with lentiviral vectors expressing shRNAs to individual kinases. The percentages of the decrease in cell proliferation/survival normalized to a scrambled control shRNA and compared to HFKs as determined by Alamar blue assays are shown. The numbers represent averages of 2 to 4 independent experiments, each performed in quadruplicate. CxCa represent averages of the 3 cervical carcinoma lines tested. Only kinases that show average differences of ≧50% (CxCa and HPV-immortalized HFKs) or ≧40% (HPV-oncogene expressing HFK populations) are shown.

FIG. 2A is a series of line graphs showing that multiple PAK3 and SGK2 lentiviral shRNA expression vectors inhibit proliferation/viability of CaSki, SiHa and HeLa cervical carcinoma cells more efficiently than in primary human foreskin keratinocytes (HFK) at multiple concentrations. Cell proliferation/viability was assessed by Alamar blue staining

FIG. 2B is a bar graph showing that multiple PAK3 and SGK2 lentiviral shRNA expression vectors cause decreases in PAK3 and SGK2 mRNA levels in CaSki cells. Messenger RNA levels were determined by quantitative reverse transcription PCR analysis at 30 hours after infection with the indicated shRNA vectors, control denotes infection with a vector encoding scrambled shRNA. Bar graphs represent averages and standard deviations of 3 independent experiments and are normalized for GAPDH expression.

FIG. 2C is a series of photomicrographs showing that multiple PAK3 and SGK2 shRNA expression vectors inhibit proliferation/viability of HPV16 E6 expressing HFKs more efficiently than matched control HFKs. Cells were stained with crystal violet and photographed.

FIG. 3A is a bar gragh and photograph of a Western blot. Human foreskin keratinocytes (HFK) transduced with control vector (HFK-c), wild type HPV16 E6 (HFK-16E6) or the p53 degradation defective HPV16 E6I128T mutant (HFK-16I128T) were infected with lentiviral vectors encoding scrambled (Control 1, 2), SGK2 specific and PAK3 specific shRNAs and cell proliferation/viability was assessed by Alamar Blue assays. A Western blot documenting p53 degradation in HFKs expressing wild type HPV16 E6 but not the HPV16 E6I128T mutant is shown on the right.

FIG. 3B is a photomicrograph, bar graph, and photograph of a Western blot. HFKs infected with a control or p53 specific shRNA expression vector (3756), were infected with shRNA expression vectors encoding scrambled, SGK2, PAK3 or MAP3K8 specific shRNAs. Photomicrographs are shown in the left. panel, a Western blot documenting p53 depletion is shown in the middle panel and quantification of Alamar blue assays are shown in the right panel. The data show that inhibition of cell proliferation/viability by SGK2 and PAK3 depletion is related to loss of p53 tumor suppressor activity.

FIG. 4A is a photomicrograph and a bar graph. Primary human mammary epithelial cell infected with a control or p53 specific shRNA expression vector were infected with shRNA expression vectors encoding scrambled, SGK2 or PAK3 specific shRNAs. Photomicrographs are shown in the left panel and quantification of Alamar blue assays are shown in the right panel. FIG. 4B is a bar graph. Primary human prostate epithelial cells infected with a control or p53 specific shRNA expression vector were infected with shRNA expression encoding scrambled, SGK2 or PAK3 specific shRNAs. Quantifications of Alamar blue assays are shown. The data show that depletion of p53 causes synthetic lethality with SGK2 and PAK3 loss in primary human epithelial cells derived from multiple tissues.

FIG. 5 is a photomicrograph showing that depletion of SGK2 and PAK3 in HeLa cells is associated with autophagy and apoptosis, respectively. HeLa cells were infected with lentiviral vectors encoding scrambled (right panels), SGK2-specific (middle panels) and PAK3-specific shRNAs (right panels). Cells were stained with antibodies for the autophagy marker LC3 (upper panels) and active caspase 3 (lower panels) and counterstained with Hoechst 33258 and phalloidin dyes to visualize nuclei and actin cytoskeletal structures, respectively.

FIG. 6 is a photograph of a Western blot showing expression of HPV16 E7, pRB and p53 in HFK populations. Decreases in p53 and pRB steady state levels served a surrogate marker for HPV16 E6 or E7 expression, respectively.

FIGS. 7A-D are tables showing the results of essential kinase screens performed with cervical carcinoma and primary human foreskin keratinocyte (HFK) cells. Cells infected with the indicated kinase specific lentiviral shRNA expression vectors were assessed for cell viability using Alamar Blue. Percent viability was normalized to cells infected with a scrambled (SCRAM) shRNA control vector. The average percent loss of viability determined for each cell line, calculated from two to four independent shRNA screens each performed in quadruplicate, is given for each shRNA expression vector tested. Ave CxCa and Ave HKF denote the average percent loss of viability in the cervical cancer lines and the two independent primary human foreskin keratinocyte (HFK) populations, respectively. Percentages of difference in viability of cervical cancer lines as compared to HFKs is also listed.

FIGS. 8A-D are tables showing the results of essential kinase screens performed with cervical carcinoma cell lines (CxCa), late passage (HKc/DR) and early passage (HKc/HPV16) HPV16 immortalized keratinocyte lines and passage/donor matched keratinocyte populations expressing the HPV16 early coding region (16ER), HPV16 E6/E7 (16E6/E7), E6 (16E6) or E7 (16E7). Cells infected with the indicated kinase specific lentiviral shRNA expression vectors were assessed for cell viability using Alamar Blue. Average percent viability calculated from two to four independent shRNA screens each performed in quadruplicate was normalized to cells infected with a scrambled (SCRAM) shRNA control vector. Percentages of difference in viability compared to HFKs are listed for each cell population tested.

FIGS. 9A-T is a series of diagrams showing grouped structures of PAK3 inhibitory compounds: Chemotypes 1, 2, 3, 3a, 3b, 3c, 3d, 4, 5, 6, 7, 8, 8a, 9, 10, 11, 12, 13, 14, and “singletons”, respectively.

FIG. 10 is a series of diagrams showing Markush structures of derivative compounds (PAK3 inhibitors) based on the basic structure of Chemotypes 4, 5, 8, 8a, 12, and 13 of PAK3 inhibitors.

FIG. 11 is a series of diagrams showing a structures of SGK2 inhibitory compounds grouped in Chemotypes 1, 1a, and 1b.

FIG. 12 is a series of diagrams showing a structures of SGK2 inhibitory compounds grouped in Chemotypes 2 and 2a.

FIG. 13 is a series of diagrams showing a structures of SGK2 inhibitory compounds grouped in Chemotype 4.

FIG. 14 is a series of diagrams showing a structures of “singletons” (SGK2 inhibitory compounds).

FIG. 15 is a series of diagrams showing Markush structures of derivative compounds (SGK2 inhibitors) based on the basic structure of Chemotypes 1, 1b, and 2a of SGK2 inhibitors.

FIG. 16 is a diagram of a synthetic scheme for an SGK2 inhibitor.

FIG. 17 is a diagram of a synthetic scheme for an SGK2 inhibitor.

FIG. 18 is a diagram of a synthetic scheme for an SGK2 inhibitor.

FIG. 19 is a diagram of a synthetic scheme for an SGK2 inhibitor.

FIG. 20 is a diagram of a synthetic scheme for an SGK2 inhibitor.

FIG. 21 is a diagram of a synthetic scheme for an SGK2 inhibitor.

FIG. 22 is flow chart showing a process for identifying kinases required for human cell proliferation and viability.

FIG. 23 is a diagram showing HPV genes and stages of cervical carcinogenesis.

### DETAILED DESCRIPTION OF THE INVENTION

The present invention provides methods and compositions for reducing, inhibiting or preventing cell proliferation and/or killing tumor cells, e.g., tumor cells in which p53 is inactivated.

p53 Association with Cancer

Germline mutations of the p53 gene are associated with some inherited cancers. Somatic p53 genetic mutations have been shown to be involved in tumors of the anus, bone, bladder, brain, breast, colon, cervix, esophagus, stomach, liver, lung, lymphoid system, ovary, prostate and skin.

In lung cancer, a mutagen found in cigarette smoke binds to DNA and ultimately can cause G (guanine) to T (thymine) substitutions in DNA. Other chemicals in cigarette smoke have been shown to produce C (cytosine) to A (adenine) changes. When these occur in the p53 gene, the mutations can cripple the p53 protein, disrupting its tumor-suppressing function.

Two major causes of liver cancer are infection with the Hepatitis-B virus and exposure to aflatoxin, a mutagen produced by a mold that grows on improperly stored grains and food crops, specifically wet corn. Aflatoxin, like benzopyrene, may alter the gene that encodes p53, thereby disrupting the tumor-suppressing ability of p53. The Hepatitis-B virus works to inactivate p53 in a different way; it produces a protein that has the ability to bind p53 and prevent it from interacting effectively with its target genes.

In skin cancer, ultraviolet (UV) rays in sunlight can cause damage to DNA. If the DNA in a skin cell is damaged beyond repair, the p53 protein can induce cell death. However, if the UV light causes a mutation in the p53 gene rendering the protein nonfunctional, the damaged cell may reproduce and potentially lead to the formation of a cancerous growth.

HPV is a sexually transmitted virus that can infect cervical cells. Once inside the cell, the virus produces a protein that binds to p53 and causes the p53 protein to be degraded. The result of this degradation is a decrease in available p53 protein and a loss of functional p53 activity.

In many breast cancers, the p53 gene appears to be normal. However, in some cases the protein MDM2 is enhanced in the cells and binds to the p53 protein, inhibiting its antitumor activity. This allows for the growth of malignant breast cells and inhibits the p53 induced apoptotic pathway.

Thus, p53 is implicated in cancers of the bladder, brain, breast, cervix, colon, esophagus, larynx, liver, lung, ovary, pancreas, prostate, skin, stomach, and thyroid. Among common tumors, with 60% of colorectal cancers, 70% of lung cancers, and 40% of breast cancers carry p53 mutations. p53 is also linked to cancers of the blood and lymph nodes, including Hodgkin's disease, T cell lymphoma, and certain kinds of leukemia. The compositions and methods of the invention are useful to treat the foregoing tumor types.

### HPV and Carcinogenesis

Papillomaviruses are small double stranded DNA viruses. Subtypes HPV-16 and HPV-18 cause cervical cancers. HPV viral oncoproteins E6 and E7, transform cells and are necessary to maintain a malignant phenotype. If E6 and E7 are removed, cervical cancer cells die. Both E6 and E7 bind to and inactivate cellular targets such as tumor suppressor proteins p53 and retinoblastoma (Rb). The HPV model of cancer progression is well characterized at a molecular level, with E6 and E7 expression being causative agents at early stages of carcinogenesis. Experiments were therefore carried out to identify kinases required at various stages of carcinogenesis, e.g., after E6 expression, after E7 expression, after immortalization, after transformation, and at various stages of cervical carcinoma development.

Functional Screen for Kinase Requirements.

Loss of function screens were carried out to determine kinase requirements in different cell lines using a lentivirus vector system that produces shRNA targeting kinases. Loss of function shRNA screens determined kinase requirements in human cell lines. Cells were transduced with a lentiviral shRNA library that targeted kinase family members, and cell lines were compared to evaluate growth inhibition. Downregulation of the same kinase was found to have a different effect depending upon the cell lines. For example, in one cell line, loss of a certain kinase had a minor effect on cell growth. However, in another cell line, loss of the same kinase was found to have a profound effect on cell growth, i.e., a much greater growth inhibitory effect was observed. Thus, downregulation or inhibition of the same kinase has different effects in different cells.

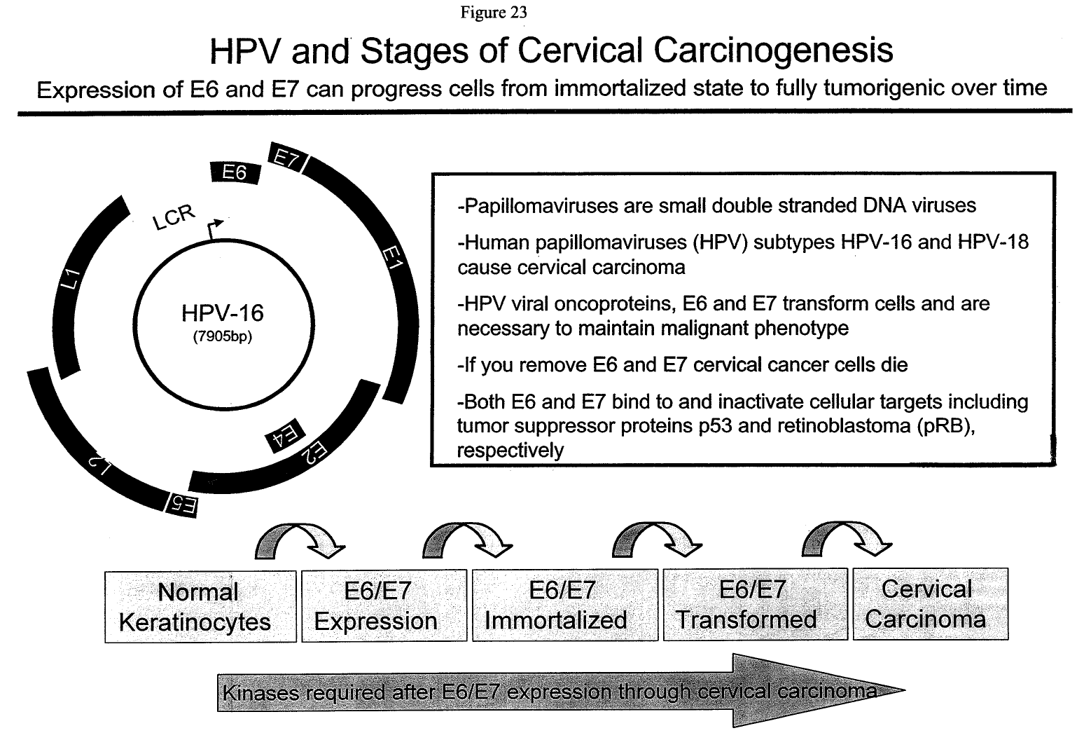

Screens were conducted to identify kinases that are required for proliferation and viability of human cells (FIGS. 23-24). A human cervical cancer cell line (HeLa) and a renal cancer cell line (293T) were screened using an RNAi library that targeted 88% of the kinome. The screen identified 100 shRNA hits that inhibited growth (greater than or equal to 50% inhibition) in either HeLa, 293T, or both cell lines (100 hits). These hits were then evaluated in 37 cell lines, including HPV oncoprotein expressing primary cells, cervical cancer cells, renal cancer cells in the absence and presence of the VHL tumor suppressor gene, breast cancer cells, and matched normal control cells of different tissues. The 100 hits represented 88 unique kinases. Some genes scored two shRNAs (e.g., ERRB3, Pak3), and others scored three or four (e.g., Jnk3 scored four shRNAs. The results revealed various cell lines downregulated for these genes showed different kinase signatures.

The essential kinase signatures were found to be remarkably different when comparing cell lines representing various tumor types, and similarities are detected only in particular settings. For example, comparison of primary cells from the same tissue and of the same lineage, irrespective of the individual donor or the date of collection yields a very similar pattern of kinase requirements. Comparison of cells that are identical except for the expression of a single gene, for example an oncogene or a tumor suppressor gene, reveals distinct changes in kinase requirements, allowing the identification of key changes in cell metabolism that are mediated by the gene in question. Whereas most tumor cells, even those isolated from the same site had different kinase requirements, we discovered a limited number of examples of tumor cells from the same site with closely related patterns of kinase requirements. In particular, the HPV18 positive adenocarcinoma cell line HeLa and the HPV16 positive squamous cell carcinoma line CaSki were amongst the most closely related tumor cell lines, whereas the HPV16 positive squamous cell carcinoma line SiHa showed a more distinct pattern of kinase sensitivity.

In addition to the kinase profiling described above, focused screens were conducted to identify kinases that are involved at various stages in HPV-associated disease. The 100 hits (WO 2007/044571 A2) were screened using HPV oncogenes (e.g., HPV-16 oncogenes) and normal foreskin keratinocytes, normal keratinocytes expressing HPV oncoprotein E6, normal keratinocytes expressing E7, normal keratinocytes expressing both E6 and E7, normal keratinocytes expressing the entire early region of the virus (E6, E7, and other proteins, and cervical cancer cells to identify shRNAs that inhibit growth of oncoprotein expressing cells and cancer cells compared to controls. Expression of E6 downregulates p53, E7 downregulates RB, and both together as well as the entire early region can downregulate both p53 and RB.

CDK7, PAK3, and SGK2 shRNAs were found to be more effective at inhibiting growth in all three oncoprotein expressing cell lines compared to normal keratinocyte control cells. SGK2 showed the most pronounced differential. Numerous cell lines were tested, and downregulation of CDK7, PAK3 and SGK2 led to enhanced growth inhibition at early stages of immortalization and at later stages of carcinoma. These results indicated the synthetic lethal interactions exist between p53 and several protein kinases and that loss of p53 makes cells reliant on novel kinases for survival. p53 loss makes cells dependent on SGK2 and PAK3, e.g., primary epithelial cells that lose p53 become dependent on SGK2 and PAK3. p53 loss changes the regulation of epithelial cells and induces the requirement for the kinases such as SGK2 and PAK3, and cells with non-functional p53 require the kinases SGK2 and PAK3.

### SGK2

SGK2 is a serine/threonine protein kinase. Although the gene product is similar to serum- and glucocorticoid-induced protein kinase (SGK), this gene is not induced by serum or glucocorticoids. This gene is induced in response loss of p53 as a result of mutation or HPV infection.

SGK kinases are members of the “AGC” subfamily (which includes protein kinase A (PKA) protein kinase B (PKB, and protein kinase G (PKG)), and there are three SGK isoforms. The serum- and glucocorticoid-inducible kinase 1 (SGK1) was the first cloned, and originally found to be an early response gene that was transcriptionally activated by serum and glucocorticoids. SGK2 kinase is closely related (80% homology) to SGK1 and SGK3, in addition to showing 54% homology to protein kinase B (AKT) in its catalytic domains. The SGK kinases become activated and function through their phosphorylation by PI 3-kinase family members, including the 3-phosphoinositide (PIP3)-dependent kinase PDK1. SGK1 is phosphorylated at one major site in vitro by PDK1, and SGK2 and SGK3 kinases are phosphorylated at two major sites, including a Thr residue in the activation loop and a Ser in a hydrophobic motif. Like PKB and SGK1, the substrate specificity of SGK2 and SGK3 involves the phosphorylation of Ser and Thr residues that lie in Arg-Xaa-Arg-Xaa-Xaa-Ser/Thr motifs. SGK1 function plays an important role in activating potassium, sodium, and chloride ion channels, and plays a role in regulating processes such as cell survival, neuronal excitability, and renal sodium excretion. The SGK1 gene contains p53-binding sites in its promoter.

### PAK3

PAKs, e.g., PAK3 are also serine/threonine protein kinases. These kinases bind to and, in some cases, are stimulated by activated forms of the small GTPases, Cdc42 and Rac. PAK3 was also found to be induced in response loss of p53 as a result of mutation or HPV infection.

PAK3 is a serine/threonine protein kinase that belongs to the “STE” subfamily and there are six PAK isoforms. PAKs are key regulators of cancer signaling pathways. PAK1 is the best characterized member and was originally identified as a protein that interacts with CDC42 and RAC1, which are members of the Rho GTPase family of proteins. The GTPase-activated PAKs localize to the leading edge of cells and function to stimulate cell motility and invasion. Increased PAK1 expression and/or activity have been linked to several cancers including breast, colon, ovarian, bladder, brain and T-cell lymphomas (Kumar et al., 2006, Nat. Rev, Cancer. 459-71.). Increased PAK4 expression has been confirmed in pancreatic cancers. All six PAK genes carry p53 consensus binding sites in their promoters.

PAK3 inhibition is synthetically lethal in combination with expression of a single HPV oncogene, E6, in primary human epithelial cells. The major cellular target of E6 is p53, which is targeted for proteasome-mediated degradation upon binding to E6. PAK3 inhibition leads to preferential cell death in cells that have lost p53 tumor suppressor activity.

### CDK7

This protein forms a trimeric complex with cyclin H and MAT1, which functions as a Cdk-activating kinase (CAK). It is an essential component of the transcription factor TFIIH, that is involved in transcription initiation and DNA repair. This protein is thought to serve as a direct link between the regulation of transcription and the cell cycle. CDKs are phosphorylated within the activation segment (T-loop) by a CDK-activating kinase (CAK) to achieve full activity. As with the other kinases described above, CDK7 is induced in response loss of p53 as a result of mutation or HPV infection.

The following materials and methods were used to generate the data described herein.

### Methodology

Experiments were performed using several control lentiviruses (those expressing GFP, expressing scrambled shRNAs, and expressing shRNAs for commonly required kinases are always included in our tests), and procedures were thoroughly assessed and validated.

Relative viral titers were tested by comparing the levels of puromycin-N-acetyl transferase (PAC) sequences in the virus stocks. PAC sequences in the viral stocks were found to be within two-fold of one another and therefore not significantly variable. Additionally, a test plate with negative control viruses from each batch is tested for accurate viral titers of drug-resistant colonies following transduction of test mouse cells. Parallel cultures of GFP-expressing lentiviruses yielded similar levels of fluorescence following infection.

Single doses of lentivirus shRNAs measured at a single time-point show differences in their responses among cell lines. To test the differences more quantitatively multiple cell lines (HFKs, HFKs+E6 and cervical cancer cell lines) were used in more comparative screens, assaying for preferential killing of oncoprotein expressing cells and cancer cells over the normal HFKs. First, a time course of shRNA knockdown was used to study the specific kinases required for cell proliferation. Although each targeted kinase mRNA exhibits its own decay curve and subsequent individual protein degradation time, Day 6 post-infection was determined to be the best point to compare the effects of shRNA expression. Second, a viral titration was performed and used to deliver shRNAs to cells over a wide range of viral MOI's. Viral transductions were done with different dilutions of virus supernatant. AlamarBlue readings were made at 6 days post infection and values were normalized to the non-killing scrambled control shRNA and converted to percent reduction in viability.

Experiments were also performed to demonstrate that differential effects observed between cell lines were due to specific down-regulation of kinase mRNAs, and that down-regulation occurred irrespective of the functional outcome. Various shRNAs homologous to different regions of particular kinase mRNAs were used to demonstrate that similar phenotypes are induced upon infection. Multiple shRNAs do show similar phenotypic changes, making it unlikely that the resulting phenotypes were due to off-target effects.

Time courses of mRNA decay were also performed, and similar decay profiles were seen in both cell lines, despite different levels of cell survival. These data suggest that siRNA machinery works similarly in different cell lines. Additionally, GAPDH mRNA levels were measured during various time-points post-infection with the lentiviruses. GAPDH levels were found to not significantly change during infection, illustrating that decay of kinase mRNA levels is due to siRNA action, not a consequence of impending cell death.

In another study using the 100 shRNA hits, the most detailed comparisons were performed between HeLa cervical carcinoma cells and 786-0 renal carcinoma cells. 15 kinases were identified showing differential requirements in either one tumor type or the other.

CDK4, FGFR3 and PDGFR were identified using 293T and HeLa cells. The kinases found from these studies comprise established kinase targets, which have been advanced as preclinical and clinical candidates for the treatment of cancer such as CDK4, FGFR3, PDGFRB as well as previously unknown kinase targets. These studies demonstrate the power of these comparative screens, and methods of identifying novel therapeutic targets for cancer.

### Cell Culture

Normal human foreskin keratinocytes (HFKs) were obtained from neonatal foreskins and cultured using standard methods. HPV oncogene expressing cell populations were generated by transfection of appropriate β-actin expression plasmids using nucleofection (AMAXA). HPV16 E7 expression was assessed by Western blotting; decreased p53 expression was used as a surrogate marker for HPV16 E6 expression. In some experiments, HPV16 E6I128T mutant was used. HFKs with p53 knockdown were obtained by infection with appropriate lentiviral shRNA vectors followed by selection in 2 μg/ml puromycin. Experiments were performed with donor/passage matched cells. Low (HKc/HPV16) and high passage (HKc/DR) HPV16 immortalized cells were grown in K-SFM (Gibco). HeLa, CaSki, and SiHa cells were grown in DMEM supplemented with 1% penicillin-streptomycin and 10% calf serum. Primary human mammary and prostate epithelial cells were purchased from Clonetics/Lonza and grown in the specific media supplied.

Infections with shRNA Expressing Lentiviruses

Lentiviruses expressing shRNAs were produced as previously described (40). 2,000-3,000 cells were seeded per well in 96-well plates. Cells were infected at 24 hours after plating. Viability assays using Alamar blue were performed after puromycin selection at five days post-infection. Cells were stained with crystal violet for image acquisition.

Quantitative RT-PCR. RNA was isolated using the RNeasy 96 Kit (Qiagen). Quantitative RT PCR analysis was performed using the QuantiTect SYBR Green RT [symbol] PCR Kit (Qiagen) on an Applied Biosystems 7300 Real Time PCR System. Primers were 5′-GCTCGACTATGTCAACG-3′ (forward; SEQ ID NO:1) and 5′-CCAAGAGAATGTTCTCTGG-3′ (reverse; SEQ ID NO:2) for SGK2 and 5′-CCAGATCACTCCTGAGC-3′ (forward; SEQ ID NO:3) and 5′-CCAGATATCAACTTTCGGACC-3′ (reverse; SEQ ID NO:4) for PAK3.

### Immunofluorescence

Three days post-infection with appropriate lentiviruses, cells washed with PBS, fixed for 15 minutes with 4% paraformaldehyde in PBS, permeabilized for 15 minutes with 0.2% Triton-X-100 in PBS and incubated for 2 hours with either LC3 rabbit polyclonal antibody (Santa Cruz Biotechnology) or Cleaved Caspase-3 rabbit polyclonal antibody (Cell Signaling Technology) diluted in blocking buffer consisting of 0.5% BSA in PBS. The secondary antibody was a goat anti-rabbit antibody conjugated with Alexa Fluor 488 (Molecular Probes/Invitrogen) and the final two-hour incubation step also contained rhodamine, phalloidin and Hoechst 33258 dyes (Molecular Probes/Invitrogen). Fluorescent images were acquired with an inverted fluorescence microscope (Zeiss) at a magnification of 200×.

### Reagents, Substrates and Compound Library

Recombinant human full length PAK3 enzyme was obtained (Invitrogen), immediately aliquoted and kept in storage buffer as purchased at −80° C. for long term storage. The HTRF® KinEASE™ kit (CisBio) containing 5× stock solution of the enzymatic buffer and 1× detection buffer, STK substrate 2-biotin (S2), a phosphospecific monoclonal Europium-labeled Cryptate antibody, which recognizes a phospholylation epitope of the biotinylated peptide, as donor fluorophore and Stretptavadin-linked XL665 (SA-XL665) representing the acceptor fluorophor were used for TR-FRET assays. The compound library consisted of approximately 125,000 small molecules, including compounds approved by the Food and Drug Administration (FDA), a purified natural products library and commercially available compounds from various vendors. All small molecules generally adhere to Lipinski's rules and have been optimized for maximization of molecular diversity.

### Enzyme Based Kinase Assays and Hit Identification

An enzyme based kinase assay was carried out using full length recombinant PAK3 kinase. A model biotinylated peptide RRRSLLE (SEQ ID NO:5) was used as the substrate. Detection was done by Homogeneous Time Resolve Fluorescence (HTRF) with an antibody that recognizes the phosphorylation site on the peptide.

Approximately 150,000 compounds were screened using this PAK3 assay. Compounds were assayed at 10 microM and a percent inhibition of the enzyme by the compounds were determined. Those compounds that inhibited PAK3 by >50% at 10 microM were identified as hits. The PAK3 inhibitors identified in that matter as well as derivatives thereof (which possess the same or similar kinase inhibitory activity) cause preferential cell death in cells that have lost p53 tumor suppressor activity and are therefore useful as anti-cancer agents.

TR-FRET assays were carried out as follows. A CRS CataLyst Express robotic arm and a Cybi-well 384 channel simultaneous pipettor were used to carry out the High-Throughput Screen. Kinase reactions were performed in 50 mM HEPES, pH 7.0, 0.02% NaN₃, 2 mM MgCl₂, 0.01% BSA, 0.1 mM Orthonanadate, 1 mM DTT, 0.001% Tween-20, 0.001% Brij-35 using ProxiPlate-384 Plus white assay plates. In the final HTS conditions, 3.2 nM PAK3 enzyme (1.6 nM final concentration in assay) and 0.2 μM biotinylated S2 peptide (0.1 μM peptide final, at the Kₘ) in a volume of 5 ml kinase buffer were added to dried solutions of 10 μM compound and pre-incubated for 30 minutes. The kinase reaction was initiated by addition of 5 ml of 50 mMATP per reaction (25 μM ATP final, at the Kₘ). The reaction was incubated for 30 minutes at room temperature. Reaction was terminated by addition of 10 μl of a premixed solution of EDTA, Europium cryptate-labeled antibody and fluorophore-conjugated streptavadin. After overnight incubation at room temperature, TR-FRET measurements were performed using a PHERAstar HTS microplate reader, and were expressed as ratios of acceptor fluorescence at 665 nm over donor fluorescence at 620 nm.

### Cell Based Assays

HeLa cells (ATCC) were maintained in Dulbecco's modified Eagle medium (DMEM) supplemented with 10% fetal bovine serum and 1% Penicillin/Streptomycin. Approximately 200 to 500 cells per well were seeded in a 96 well tissue culture plates. Following 24 h growth in normal DMEM, the individual compounds were warmed up to room temperature and diluted in DMEM to their according concentration and then immediately added to the wells. One column on each assay plate contained DMEM only (“neg. control”) and one column contained untreated HeLa cells as “pos. control”. Cells were treated with the compounds at various cell densities and for treatment periods ranging between 75 hrs and approx. 200 hrs. To control for cell viability, we assayed plates using AlamarBlue° (Invitrogen), which uses a redox reaction to measure metabolically active viable cells. At each time point the culture media was removed and a solution of 10% AlamarBlue reagent and DMEM was added to each well to measure cell viability. Fluorescence was measured after 1-2 hrs incubation at 37 C on a Victor² Plate Reader (PerkinElmer).

### Data Analysis

Data were analyzed using GraphPad Prism Version 5 (GraphPad Software Inc., La Jolla, Calif., USA). Each compound plate included one column of negative controls, were no enzyme was added, and another column for positive controls when no inhibitor compound was added; these were used to calculate Z′ factors and signal to noise ratios throughout the screen. Percentage of inhibition of PAK3 enzyme activity was calculated according to the following equation: % inhibition=100×(average of positive controls−test compound value)/(average of positive control−average of negative controls). IC₅₀ determinations were done in quadruplicate for each compound using different adding sequences for compound and enzyme-substrate-mix. EC₅₀ determinations were done in triplicate for each compound using different time points. For calculation of IC₅₀ and EC₅₀ concentrations, respectively, mean inhibition dose response curves were fitted to the sigmoidal response equation: Y=Bottom+(Top−Bottom)/(1+10̂((X−LogIC₅₀))) were X is log(compound concentration) and Y is % inhibition, and Bottom and Top are the lower and upper plateau. Km concentrations were determined by non-linear regression curve fitting, using the equation: Y=Vmax*X/(Km+X), where X is the substrate concentration.

### Essential Kinase Screens In Cervical Cancer Cells

To test the viability of using existing cervical cancer cell lines to identify essential kinases, C33A (HPV negative), CaSki (HPV16 positive at high copy number), SiHa (HPV16 positive at low copy number), and HeLa (HPV18 positive) cells were screened in a 96-well format. For each cell type, each well of a 96-well plate was infected with a lentiviral shRNA expression vector that knocked-down expression of an individual kinase. Cells were selected with puromycin to demonstrate the efficiency of the infection. Six days post infection, alamar blue was added to the culture media. Similar to tetrazolium salts, alamar blue measures the mitochondrial fitness of cells. This provides a quick, convenient, colorimetric read-out of cell number. A pink color in the well corresponds to higher cell confluency/cell survival, while a blue color corresponds to lower cell confluency/cell death. A range of colors in-between pink and blue can be measured in plate reader and gives the range of killing or arrest by individual kinase knock-downs. Screens were carried out using the “top 100 hits” against 88 individual kinases (identified in the previous screens described above). After screening 4 cervical carcinoma cell lines, screens were expanded to include normal primary human foreskin keratinocytes (HFKs, 8 different populations) and fibroblasts (2 different populations, HFFs). These screens were performed to identify essential kinases in cervical cancers versus normal primary human cells. Since HPV is such a unique and informative model system to study carcinogenesis, the screens were expanded to include HFKs and cell lines expressing the HPV16 oncoproteins. By expressing the oncoproteins, early targeting of host kinases was determined. Numerous cell lines and populations were tested (25 total including multiple populations of the following control HFKs, E6 expressing HFKs, E7 expressing HFKs, E6 and E7 expressing HFKs, early region expressing HFKs, control RKO colon cancer cells, RKOs expressing E7, control NOK (normal oral keratinocytes), NOKs expressing E7, and control HFFs).

The range of cell survival/death and efficiency of the lentiviral infection for each screen performed using the above cell lines was analyzed by plotting the average Alamar blue values from each run. Each cell line was screened in at least two independent experiments, each done in duplicate with +/−puromycin selection. Hence, each experiment was effectively done in quadruplicate. Every point on the graph represents the value of each average alamar blue value and directly corresponds to the level of cell death (for example, a blue, empty well would likely have a reading from 1,000-10,000, whereas a pink, confluent well would have a reading of 30,000-50,000 relative fluorescent units). All data points generated from each viral shRNA transduction should cluster on a linear axis, in a +puromycin (X-axis) and −puromycin (Y-axis) scatterplot, indicating that viral transduction was approaching 100%. All HPV positive cervical cancer cell lines infected well as determined by the scatterplot and showed varying levels of cell survival upon particular kinase knockdown. Other cell lines were tested, including primary cells, and showed similar plots indicating that they were also amenable to kinase screens.

Studies were carried out to determine whether ectopic expression of a single viral oncoprotein in a specific cellular background would alter kinase sensitivity. Cell lines expressing HPV16 E7 were screened, including a colorectal carcinoma cell line (RKO) and normal, hTert immortalized oral keratinocytes (NOKs). These screens showed differential killing in response to several kinase knock-downs with E7 expression. Several of the kinases identified in these E7-based screens fit the known role of E7 to inhibit the retinoblastoma tumor suppressor protein. For example, CDK6, a kinase that phosphorylates and inactives pRB, was essential in cells without E7. When E7 was present, the shRNAs for CDK6 had no affect.

Additional experiments were done to identify kinases targeted by particular oncogenes in normal, primary human foreskin keratinocytes (HFKs). The HPV16 oncogenes were expressed individually, together and in the context of the entire early region in normal, primary HFKs. Control populations expressing the empty vector were also generated. Two populations of HFKs were transfected, and cells with stable expression of HPV genes were made by G418 selection. These cells were screened at passage 5, prior to immortalization. Performing the screens in this timescale gave rise to perfectly paired control cells for comparison. The full collection of tested cells permitted interrogation at several of stages of cervical cancer development starting from normal primary cultures, to HPV expressing cells, immortalized HPV expressing cells, tumorigenic HPV-expressing cells, and the cell lines isolated from HPV-associated carcinomas. These cells were used to identify kinases that become required as cells progress through these stages of tumor development.

Percent killing was calculated for all cell lines screened by normalizing alamar blue values to those with the scrambled shRNA control. Analysis of percent killing was performed, and the shRNAs against kinases demonstrating the largest percentage difference in killing between cervical cancer cell lines and normal cells were determined. Kinase knockdowns leading to a high percent of cell death in cervical cancer cells but demonstrating a low percentage of cell death in normal cells are of the utmost interest for the development of therapeutic targets, as they are the most likely to be effective at killing tumor cells without harming normal cells. Several targets identified in the screens were also identified as essential kinases in other tumor cell lines tested.

The patterns that have emerged from these indicate that 3 kinases, CDK7, PAK3, and SGK2 were required for proliferation of cervical carcinoma cells, but not in normal primary keratinocytes. They become required in cells in all stages past the expression of HPV early proteins, and expression of E6 alone is both necessary and sufficient to establish their need in cells. Although the data indicate that CDK7 is a member of this class of kinases, the strength of its response was less than that of SGK2 and PAK3.

The action of HPV E6 proteins changes cell metabolism in such a way as to make keratinocytes now require the action of these kinases. All three cervical carcinoma cells tested rely on the independent action of these kinases, and multiple populations of primary keratinocytes development dependence on these kinases following E6 expression. Surprisingly, the inhibition of p53 by the HPV E6 protein induced dependence on SGK2 and PAK3. This observation has been borne out by further experimentation. SGK2 and PAK3 knockdowns using multiple shRNAs for each kinase and in repeated experiments had no effect on the fate or rate of proliferation of primary keratinocytes. However, the expression of HPV E6 but not mutations of E6 that fail to degrade p53 induced dependence on SGK2 or PAK3 in primary keratinocytes. Further, loss of p53 by either of two shRNAs induced dependence on SGK2 or PAK3. Finally, expression of a dominant negative version of p53 that functionally inactivates this protein similarly induced SGK2 and PAK3 requirements. These affected cells die by either apoptosis or autophagy. This phenomenon was not restricted to keratinocytes; primary mammary epithelial cells, prostate epithelial cells, and foreskin fibroblasts responded similarly. SGK2 and PAK3 mRNAs are also lowered dramatically by their cognate shRNAs.

These data establish a clear genetic interaction between p53 loss and either SGK2 or PAK3 loss. p53, SGK2, or PAK3 alone can be removed in multiple primary (normal) cell cultures with no apparent effects. However, the combination of p53 and SGK2 loss or the combination of p53 and PAK3 loss leads to cell death. These interactions then are synthetically lethal. These relationships are induced by cancer mutations, and are exploited as described herein to identify cancer targets and therapeutic agents that inhibit those targets to kill or decrease the proliferation of tumor cells with little or no adverse effect on normal non-tumor cells.

### Synthetic Lethal Interactions Between p53 and the Protein Kinases SGK2 and PAK3

Studies were carried out to determine how kinase requirements change during tumor development. SGK2 and PAK3 become essential for cell proliferation/viability as primary epithelial cells loose p53 tumor suppressor activity. Since loss of p53 tumor suppressor activity is the most common hallmark of human tumorigenesis, the identification of these kinases represent a unique class of chemotherapeutic targets—proteins that become essential following cancer mutations that may not themselves be mutated directly.

Kinases that are Essential for Proliferation/Survival of HPV-Positive Human Cervical Cancer Cell Lines

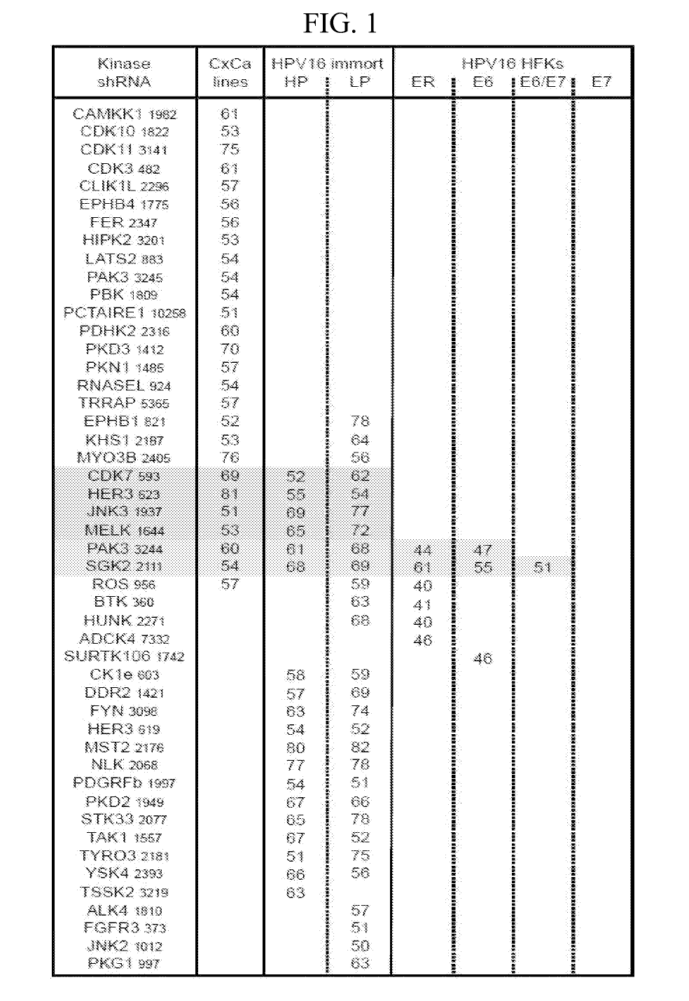

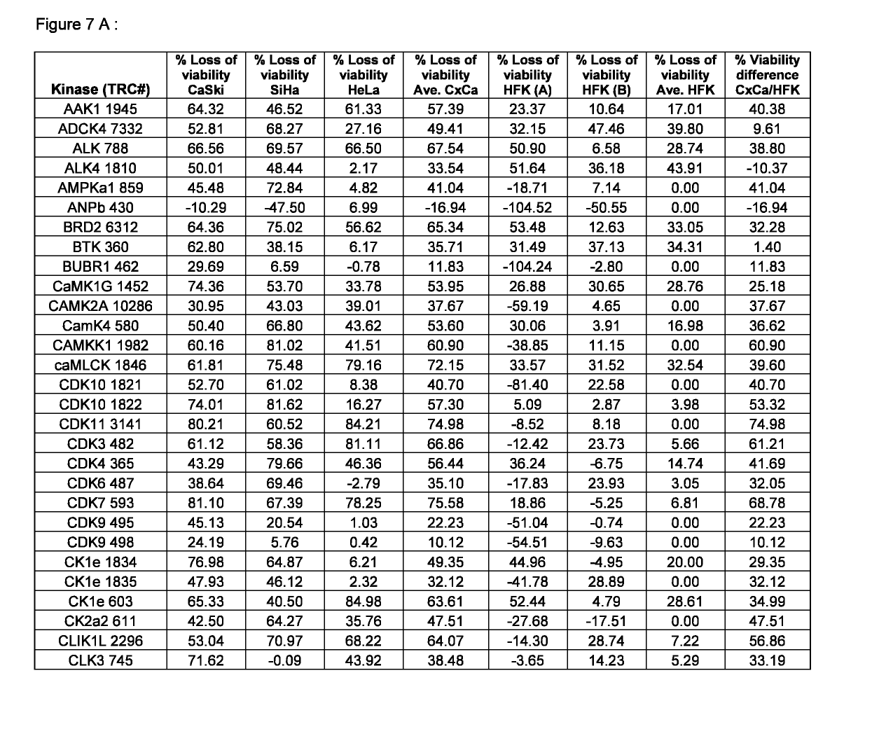

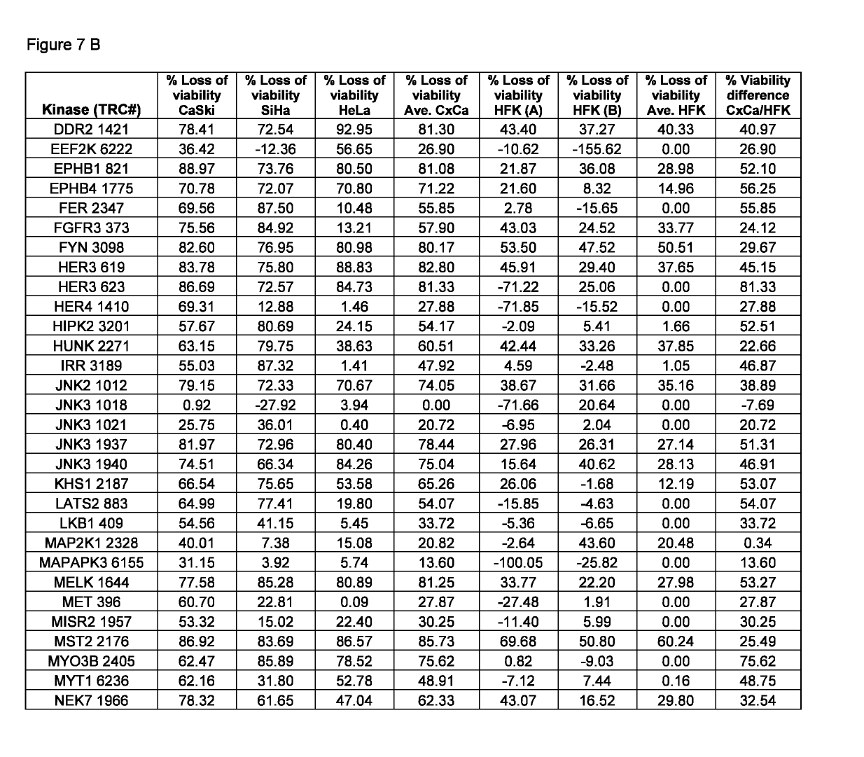

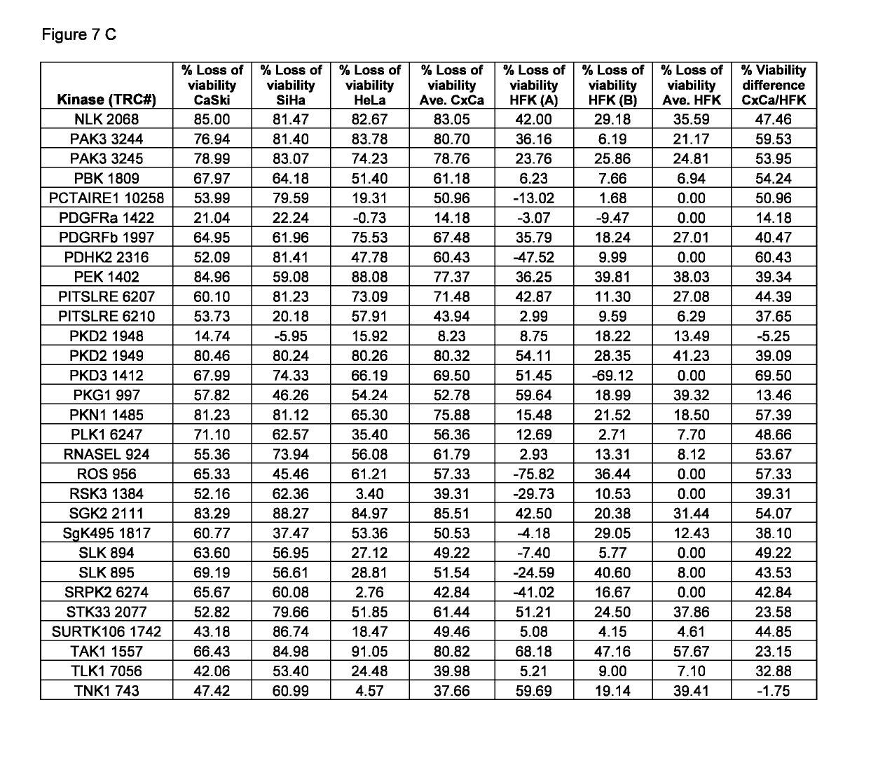

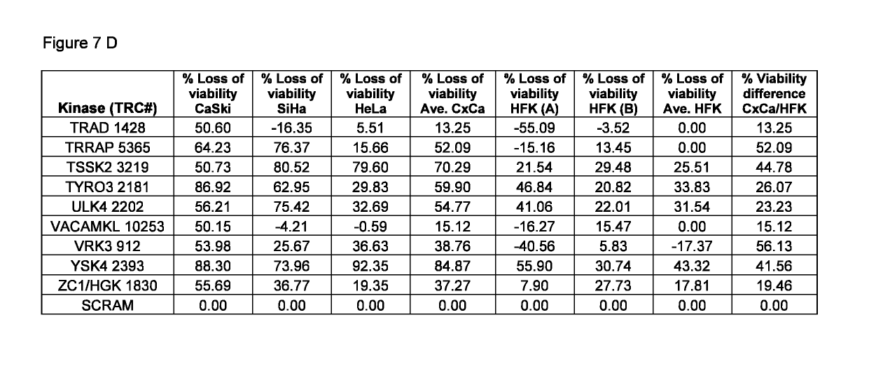

Experiments were carried out to determine whether there was a common set of kinases that were essential for proliferation/survival of three cervical carcinoma cell lines but were dispensable for primary human foreskin keratinocytes (HFKs). Cells were infected with the appropriate lentiviral shRNA expression vectors, and cell proliferation/survival was assessed by Alamar blue staining Alamar blue is a redox-sensitive dye that interrogates mitochondrial fitness of cells, and these assays provide a readout for cell proliferation/viability. The raw values were normalized to a scrambled control shRNA and are presented as % decrease in proliferation/viability. Kinases were designated “essential” (1) when an shRNA inhibited cell proliferation/viability ≧50% on average in the three cervical cancer lines, and (2) when the shRNA scored as ≧50% more effective in suppressing proliferation/viability as compared to the average response in two populations of HFKs. From the tested set of 86 kinases plus controls, 26 kinases (represented by 27 shRNAs) were identified that scored as essential by these criteria (FIG. 1, FIGS. 7A-D).

Human Kinases that Become Essential at Distinct Stages of HPV-Mediated Human Cervical Carcinogenesis

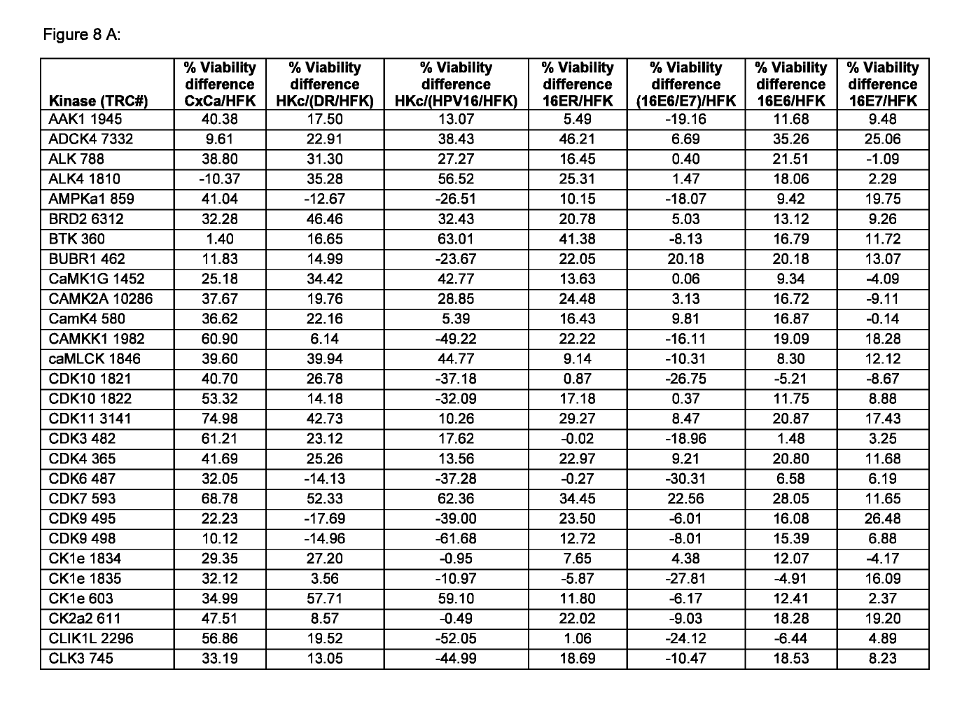

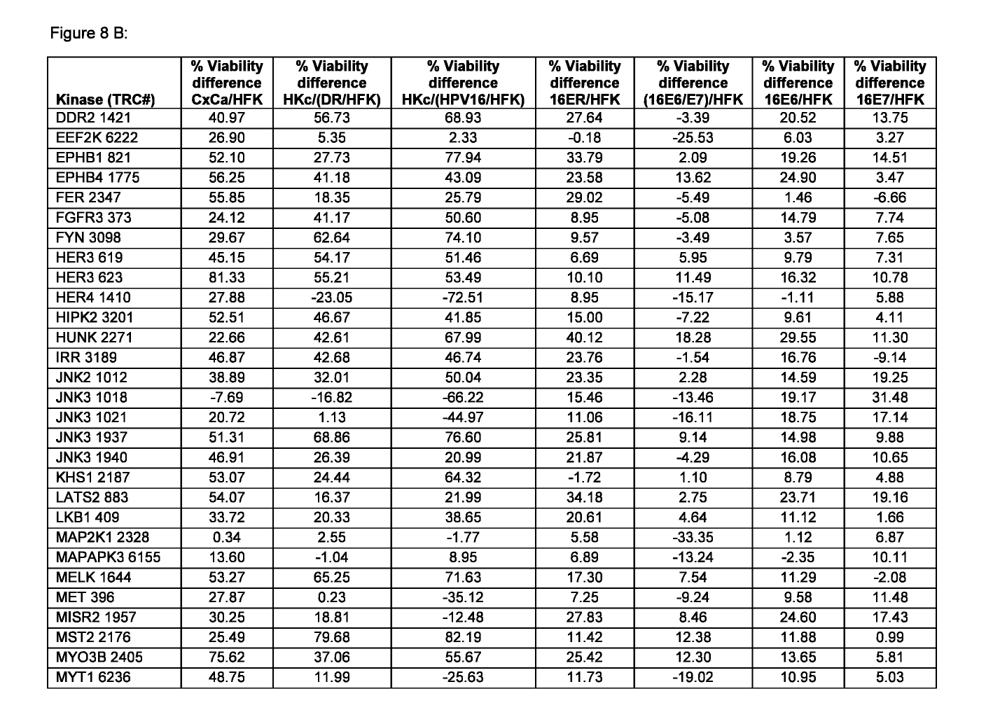

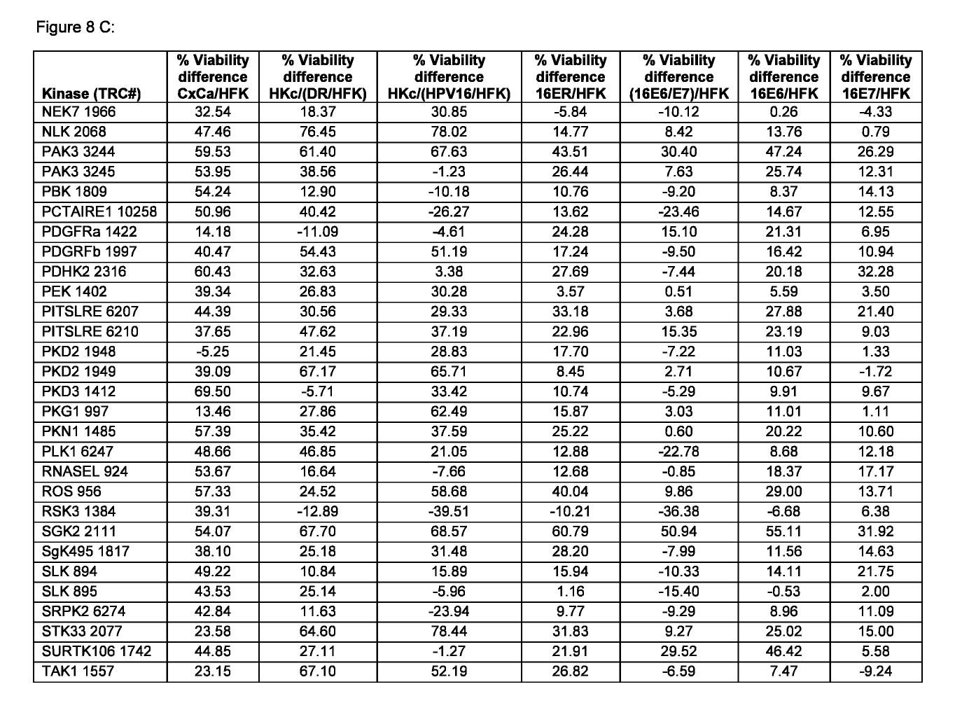

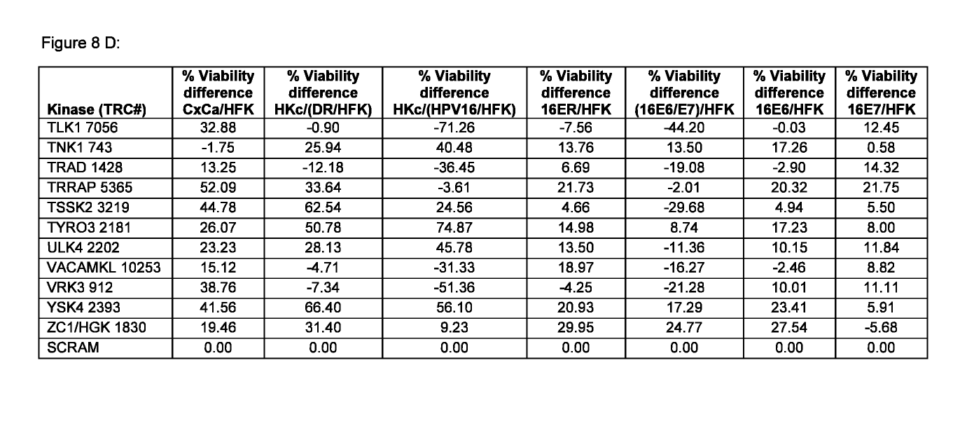

HPV-associated carcinogenesis is readily modeled in vitro using an art-recognized model system. Thus, two HPV16-immortalized HFK lines that model different stages of cervical carcinogenesis were evaluated. The two cell lines, HKc/HPV16 and HKc/DR are derived from a single piece of foreskin epithelium that was transfected with a head-to-tail dimer of the cloned HPV16 genome. Low passage cells (HKc/HPV16) represent freshly immortalized cells, whereas high passage cells (HKc/DR) have been selected for resistance to differentiation and failure to growth arrest in response to TGF-β. While both cell lines are non-tumorigenic, mRNA expression profiling studies have shown that HKc/DR are more similar to cervical carcinoma cells than HKc/HPV16 cells. As in the experiments with cervical cancer lines, kinases were identified as “essential” when their depletion yielded ≧50% difference in proliferation/survival relative to HFKs. A total of 18 essential kinases were identified for HKc/DR. Six of these, CDK7, HERS, JNK3, MELK, PAK3 and SGK2, were also essential for cervical carcinoma lines. For HKc/HPV16, 27 essential kinases were identified. Ten of these, CDK7, EPHB1, HER3, JNK3, KHS1, MELK, MYO3B, PAK3, ROS and SGK2, were also essential for cervical cancer lines. Seventeen of the 18 essential kinases for HKc/DR also scored as essential in HKc/HPV16. Six of these 17 kinases, CDK7, HER3, JNK3, MELK, PAK3 and SGK2 were essential for HKc/HPV16, HKC/DR as well as the cervical carcinoma cell lines (FIG. 1 and FIGS. 8A-D).

Human Kinases that Become Essential as a Direct Consequence of HPV Oncogene Expression

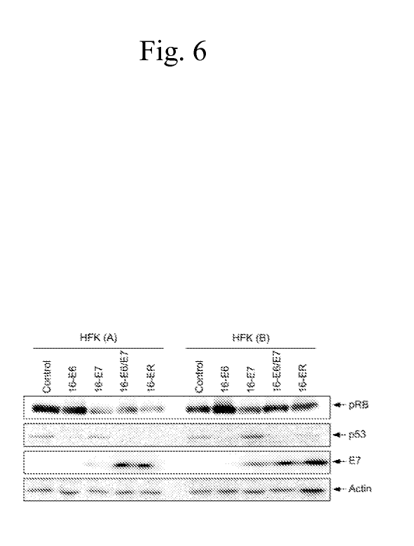

To identify kinases that become essential as a direct consequence of HPV16 gene expression, two independent sets of donor/passage matched HFK populations engineered to express the HPV16 early region or the HPV16 E6 and/or E7 oncogenes were analyzed. Expression of HPV16 E7, pRB and p53 in the corresponding HFK populations was assessed by Western blotting. Decreases in p53 and pRB steady state levels served a surrogate marker for HPV16 E6 or E7 expression, respectively (FIG. 6). Each of these HFK populations was transduced with the collection of 100 shRNAs as above. Kinases were classified as “essential” when they showed ≧40% decreased proliferation/viability relative to normal cells in each matched set. Six kinases (ADCK4, BTK, HUNK, PAK3, ROS, SGK2) met these criteria in HPV16 early region expressing HFKs, 1 (SGK2) in HPV16 E6/E7 expressing HFKs, 3 (PAK3, SGK2, SURTK106) in HPV16 E6 expressing HFKs and none scored in HPV16 E7 expressing HFKs. Whereas BTK also scored in HKc/HPV16, and ROS in HKc/HPV16 as well as cervical carcinoma cells, only PAK3 and SGK2 consistently scored as essential in HPV16 E6, early region expressing HFKs, HKc/HPV16 and HKc/DR as well as in the cervical carcinoma cell lines. These results demonstrate that HPV16 E6 expression in primary HFKs induces synthetic lethality upon loss of SGK2 and PAK3 expression, and this is retained in HFKs expressing the entire HPV16 early region, HPV16-immortalized HFKs and cervical carcinoma lines.

shRNA Targeting of SGK2 and PAK3

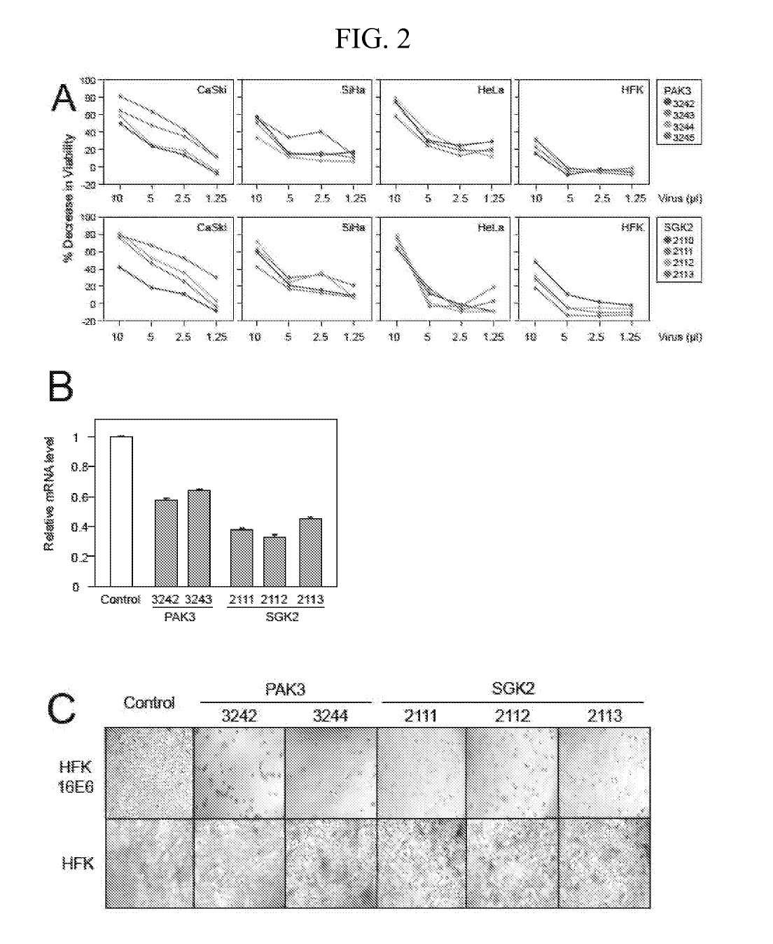

To establish quantitative comparisons of the SGK2 and PAK3 responses and determine whether additional shRNAs specific for each of the kinases yielded similar results, 4 different shRNA expressing lentiviruses for each of the 2 kinases were tested in titration experiments using CaSki, SiHa and HeLa cervical carcinoma cells and HFKs. These experiments are necessary, since infection with a single dose of an shRNA expressing lentivirus affords limited resolution, as it may not be within the linear range of the assay. These experiments revealed that multiple SGK2 and PAK3 specific shRNAs and at a variety of titers inhibited proliferation/viability in each of the cervical carcinoma lines but not in HFKs (FIG. 2A).

To confirm kinase knockdown, CaSki cells were transfected with multiple PAK3 and SGK2 specific shRNA expression vectors. Since these kinases are expressed in CaSki cells below the limit of detection by Western blotting with commercially available antibodies, mRNA levels were analyzed by quantitative reverse transcription PCR at 30 hours post infection. These experiments demonstrated significant knockdown of PAK3 and SGK2 with each of the corresponding shRNAs (FIG. 2B).

The ability of multiple PAK3 and SGK2 specific shRNAs to suppress proliferation/viability of HPV16 E6 expressing HFKs as compared to matched control HFKs was also analyzed. As shown in FIG. 2C, multiple shRNAs that target different regions of SGK2 or PAK3 mRNA inhibited cell proliferation/survival of HPV16 E6 expressing cells but did not markedly affect control HFKs.

These results demonstrate that SGK2 or PAK3 are essential for cell proliferation/viability of HPV-positive cervical cancer cell lines, HPV16-immortalized HFKs and HPV16 E6 oncogene expressing HFKs. Thus, HPV16 E6 expression causes synthetic lethality with loss of SGK2 and PAK3 expression.

### Synthetic Lethality Induced by SGK2 or Pak3 Depletion in HPV16 E6 Expressing Cells is a Consequence of p53 Inactivation

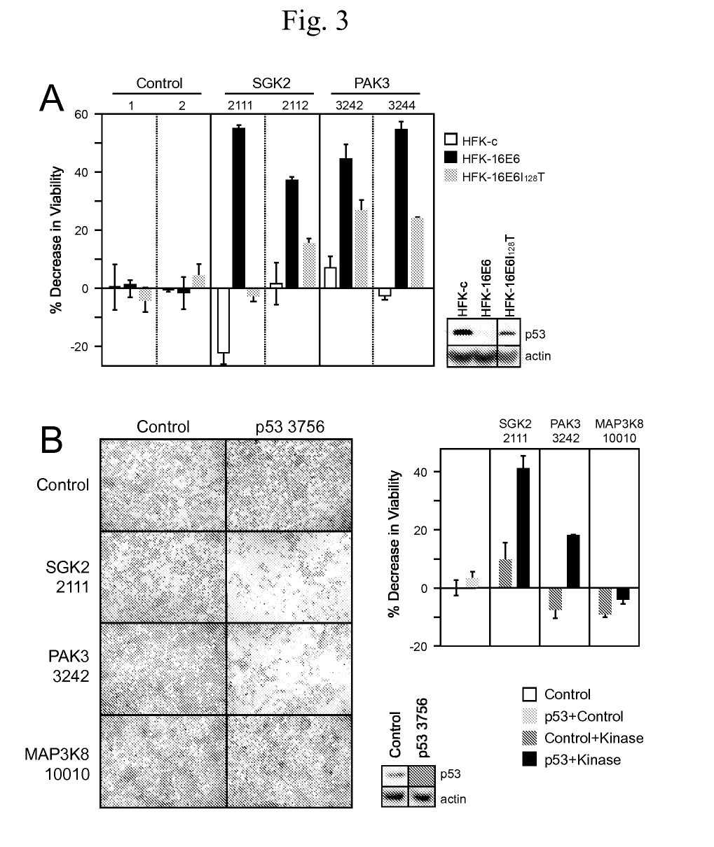

The best-known cellular target of HPV16 E6 is the p53 tumor suppressor protein. HPV16 E6 associates with the cellular ubiquitin ligase E6AP, and the E6/E6AP complex associates with p53 and targets it for proteasomal degradation. To determine whether the observed sensitization of HPV16 E6 expressing HFKs was due to p53 degradation, HFKs expressing HPV16 E6 or an HPV16 E6 I1128T mutant were generated. These cells are defective for association with the E6AP ubiquitin ligase and thus p53 degradation. Donor/passage matched vector transduced HFKs were used as controls. SGK2 and PAK3 depletion markedly inhibited cell proliferation/survival of wild type HPV16 E6 expressing cells, whereas HFKs expressing the HPV16 E6 I128T mutant were less sensitive to SGK2 or PAK3 depletion (FIG. 3A).

To directly assess the involvement of p53, p53 in HFKs was depleted by infection with a lentiviral shRNA. Co-depletion of p53 and PAK3 or SGK2 resulted in a dramatic decrease in cell proliferation/viability, whereas depletion of an unrelated kinase, MAP3K8, which does not score as synthetic lethal with HPV16 E6 expression, had similar effects in control and p53 depleted HFKs (FIG. 3B).

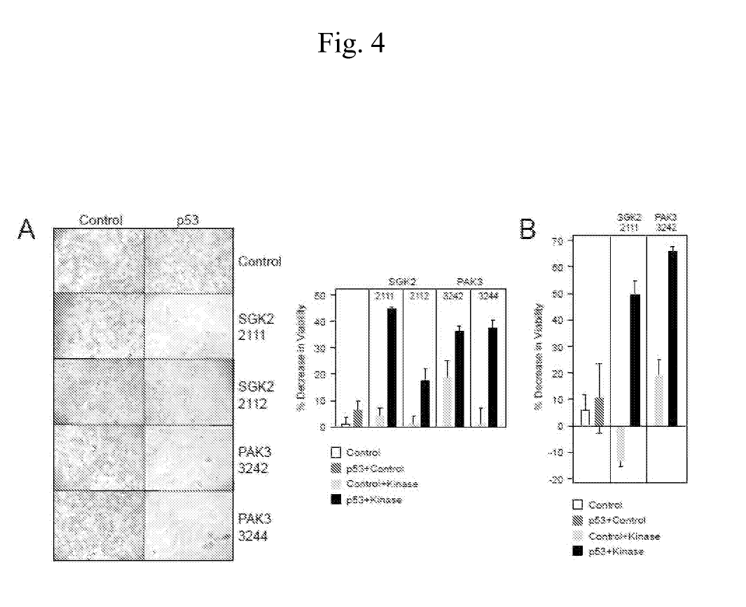

To determine whether the observed effect was specific to human foreskin derived keratinocytes or could be seen in primary epithelial cell cultures derived from other human tissues, p53 was depleted in primary human mammary and prostate epithelial cells. Similar to what was observed in the primary HFKs, p53 loss caused synthetic lethality with SGK2 and PAK3 depletion in mammary (FIG. 4A) and prostate epithelial cells (FIG. 4B). These data confirm that functional inactivation of p53 induces cellular changes that render the SGK2 and PAK3 kinases essential in primary human epithelial cells.

### Mechanisms of Synthetic Lethality

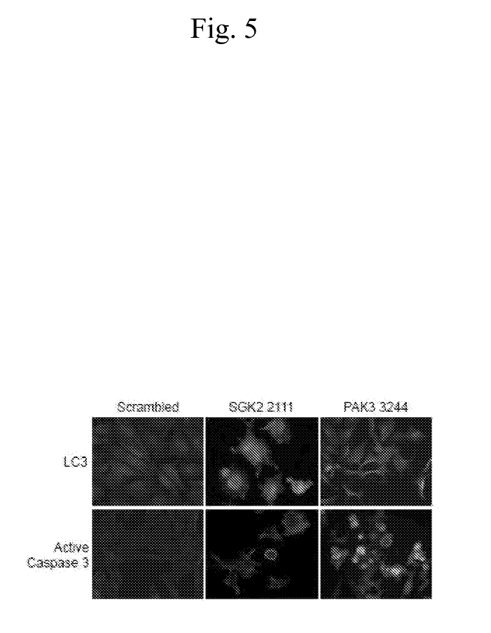

The data described herein document a block to proliferation/survival in p53-deficient cells upon depletion of PAK3 and SGK2, further studies were carried out to investigate the mechanism of action. A decrease in cell number as a consequence of kinase knockdowns may result from apoptosis, autophagy, senescence or cell cycle block. Hence, immunofluorescence experiments were carried out with antibodies for cleaved, activated caspase 3, a marker of apoptosis, and LC3, a marker of autophagy, in HeLa cells with knockdown of SGK2 or PAK3. Cells were counterstained with Hoechst and phalloidin to visualize nuclei and actin microfilaments, respectively. The data indicated that the mechanisms of synthetic lethality in HeLa cells were different for the two kinases; SGK2 depletion caused autophagy whereas PAK3 knockdown resulted in caspase 3 activation, suggestive of apoptosis. Moreover, PAK3 depletion causes marked disruption of actin filament staining, indicative of a collapse of the actin cytoskeleton, whereas no such effect was observed with SGK2 depletion (FIG. 5).

Kinase Requirements of p53-Deficient Cells

Synthetic lethal screens are one example of a larger group of genetic tests in which two genes can be shown to coordinately modify a particular phenotype and thus must have related functions within an organism. The terms “synthetic lethal” and “synthetic lethalities” were coined in 1946 by T. G. Dobzhanzky (Dobzhanzky, T., 1946, Genetics of Natural Populations. XIII. Recombination and Variability in Populations of Drosophila Pseudoobscura. Genetics 31:269-90.16). In the simplest terms, synthetic lethality is scored when either of two mutations in different genes has no effect on their own but in combination they have a lethal phenotype. Two logical premises have been proposed to explain how synthetic lethality can be achieved. In one explanation, two pathways perform redundant roles and loss of either pathway alone has no effect on the cell phenotype. However, combining the two mutations leads to a lethal phenotype by removing both pathways and depriving a cell of an essential function. In the second explanation, one protein acts upstream of the second, and loss of either has no effect. One mutation occurs in a positively acting step and the other in a negative one. Since the two proteins functionally balance one another, losing one will tip the balance slightly, but losing both is catastrophic.

Two new synthetic interactions with loss of p53 tumor suppressor activity using a limited shRNA screen were identified. The loss of SGK2 or PAK3 was lethal only when coupled to the loss of p53. The synthetic interactions between p53 and SGK2 or between p53 and PAK3 have been confirmed by several criteria. Loss of p53 through two methods; expression of the HPV E6 protein and p53 depletion cooperate with SGK2 or PAK3 loss to generate cell death. Depletion of the corresponding shRNA targets was confirmed by at the level of mRNA expression. The synthetic interactions between p53 and SGK2 loss or between p53 and PAK3 loss are not limited to foreskin keratinocytes but are seen in primary human epithelial cells from mammary or prostate tissues. The synthetic relationship between p53 and SGK2 or PAK3 is not unique to a limited cell type but is broadly applicable.

SGK2 depletion in p53 null cells leads to reduction in cell proliferation/survival via autophagy, while PAK3 depletion in p53 null cells causes apoptosis. This observation indicates that SGK2 and PAK3 are components of two independent signaling pathways that become essential following p53 loss.

Inhibitors that kill cancer cells by blocking the roles of proteins such as SGK2 or PAK3 in a p53-dependent manner but spare normal cells are useful as cancer therapeutic agents. Thus, studies were carried out to identify such agents.

### Identification of Small Molecule Inhibitors of SGK2 and PAK3

Compounds were screened using standard in vitro kinases assay. Compounds identified in this screen were further tested using HPV16 oncoprotein (e.g., E6) expressing cells compared to normal controls. Compounds that inhibited enzymatic activity in vitro were found to inhibit proliferation of p53-deficient cells.

A library of compounds was screened for inhibitors of SGK2 and PAK3 activity by assaying phosphorylation of a generic peptide substrate either directly; or indirectly by inhibiting upstream kinase PDK1 from activating the enzyme in vitro. In both cases, the phosphorylated substrate was detected using a specific anti-phospho peptide antibody that is coupled with Eu³⁺ Cryptate and XL665 conjugated with streptavidin. The initial screening concentration started at 20 μM, and the ATP concentrations were varied to determine if these inhibitors were competitive with ATP. All initial hits were re-assayed as a dose response series with eight 3-fold dilutions and resulting final concentrations ranged from 0.9 nM to 20 μM. Several hits emerged from the screen. These compounds all showed initial kinase inhibition and were dose responsive. Several hits displayed activity cell-based assays.

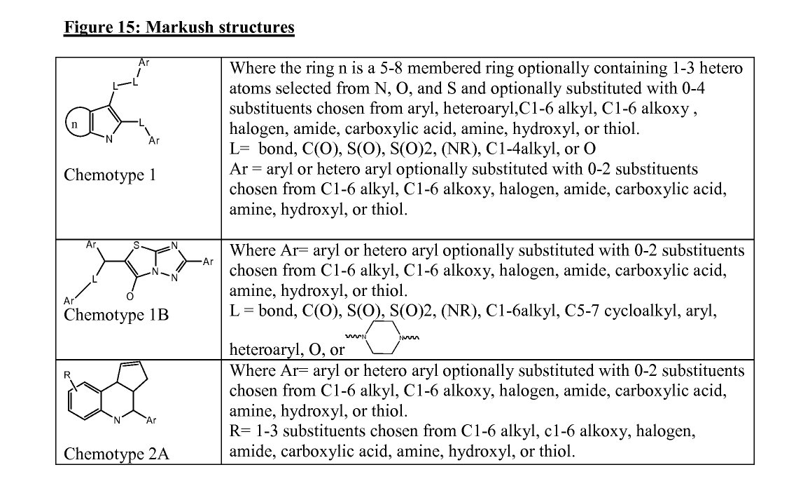

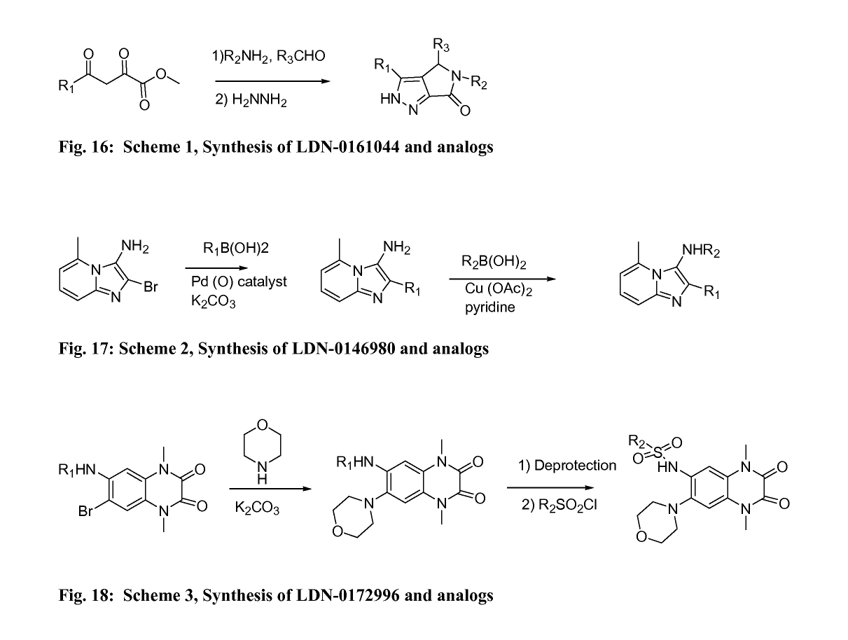

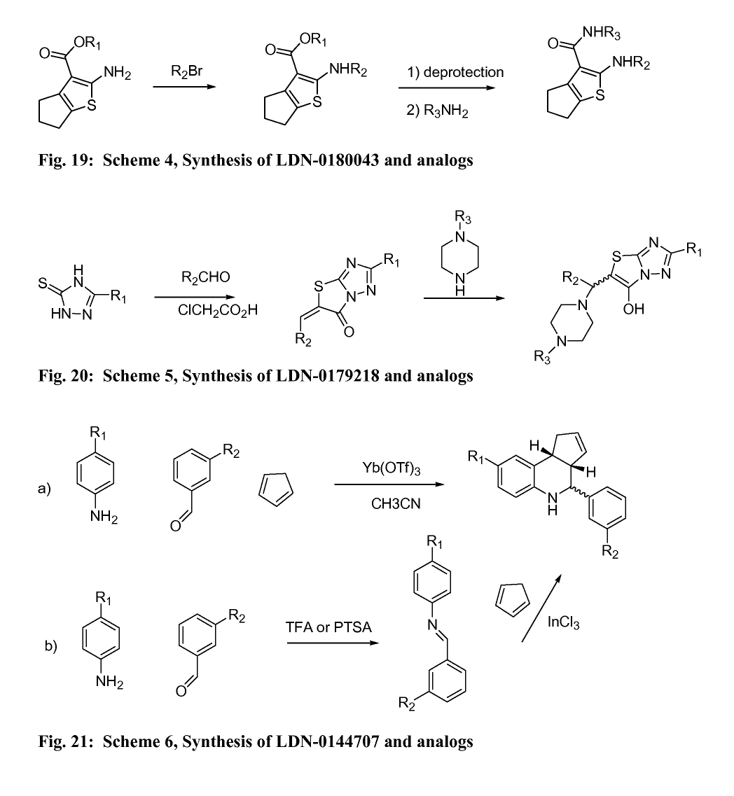

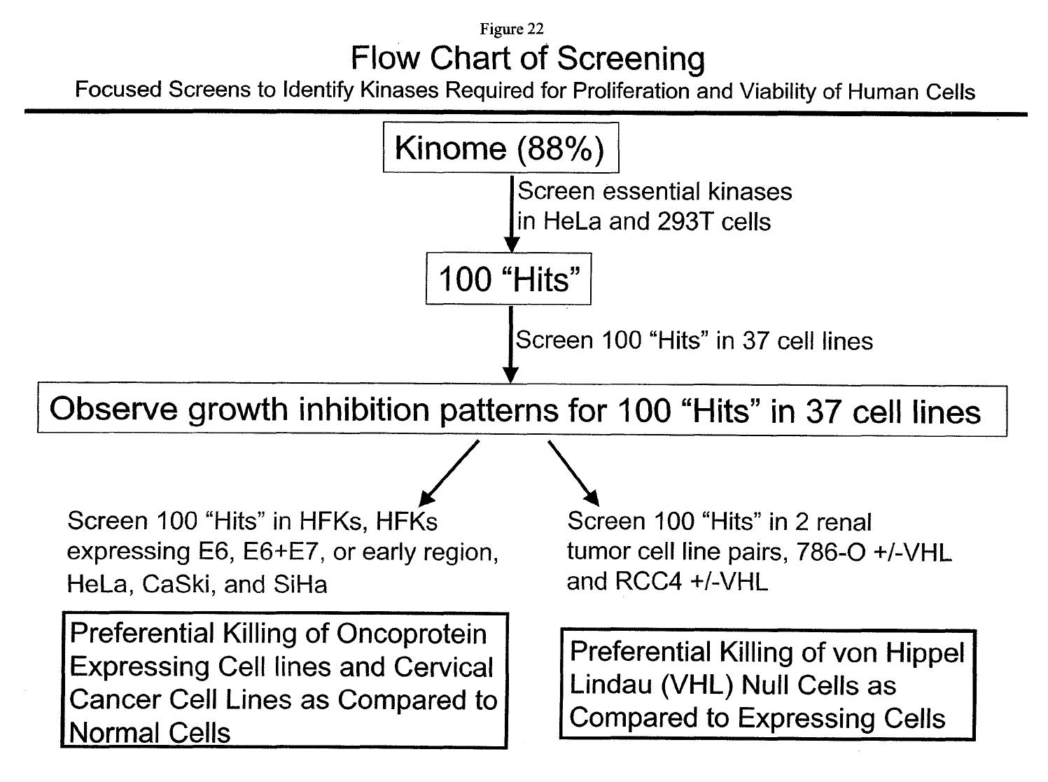

All of the small molecules (kinase inhibitory compounds) identified in the screens and described herein are synthesized using methods and reagents well known in the art of synthetic chemistry. FIGS. 16 to 21 show general synthetic schemes for the synthesis of exemplary SGK2 chemotypes. Several approaches exist for the synthesis of each chemotype. The following synthetic examples are meant to illustrate the general approach only, rather be an exhaustive synthetic search. For example, Scheme 1 outlines an approach to the synthesis of LDN-0161044. This compound and analogs can be constructed by a multi-component coupling reaction in two steps, using amines, aldehydes and hydrazine as diversity elements. Methods for the synthesis of such scaffolds are well known in the art, e.g., Zhurnal Organicheskoi Khimii, 1998, 22(8), 1749.

Scheme 2 shows the synthesis of LDN-0146980 and analogs. The synthesis is a two step process. The first step is a Suzuki reaction of the heteroaryl bromide scaffold with variety of boronic acids in the presence of palladium (0) catalyst. The second step is a copper acetate mediated coupling of a heteroaryl amine with boronic acids. Both reactions are well established transformations and variety of analogs are prepared easily.

Scheme 3 describes the synthesis of LDN-0172996 and analogs. The first step of the process is a displacement of an aryl bromide by an amine nucleophile. The same transformation is accomplished using palladium catalyzed aryl amination chemistry. Subsequent deprotection of the aryl amine and reaction with sulfonyl chloride results in the formation of the product. These reactions are well described in the literature and many analogs are prepared in an efficient manner.

Scheme 4 outlines the synthesis of LDN-0180043 and analogs. The first step is an alkylaton of an aryl amine with a bromo (or other suitable electrophile). This step is followed by deprotection and coupling with variety of amines to give the product scaffold. These reactions are well established and many analogs are prepared easily.

Scheme 5 presents the synthesis of LDN-0179218 and analogs. The process is a two step multi-component coupling reaction with aldehydes and amines as the diversity elements. General methods for the synthesis of this scaffold are well known in the art, e.g, in Archiv de Pharmazie, 1995, 328(2), 169.

Scheme 6 outlines the synthesis of LDN-0144707 and analogs and presents two general approaches. Approach (a) is a multi-component coupling reaction, based on imine formation and Diels-Alder cyclization. The diversity elements are amines, aldehydes and dienes. The process is catalyzed by Lewis acids, such as ytterbium triflate (Yb(OTf)₃. The second approach (b) is based on two discrete steps: first, the formation of an imine from the reacting aldehyde and amine and second, Diels-Alder cyclization of the imine with a variety of dienes. The reaction transformations in both approaches are well established and many analogs are prepared in an efficient manner.

### Small Molecule Inhibitors of PAK3

A medicinal chemistry evaluation of the compounds from the screen was carried out The compounds segregate into clusters and chemotypes. In addition, generic Markush structures for analogs have been defined.

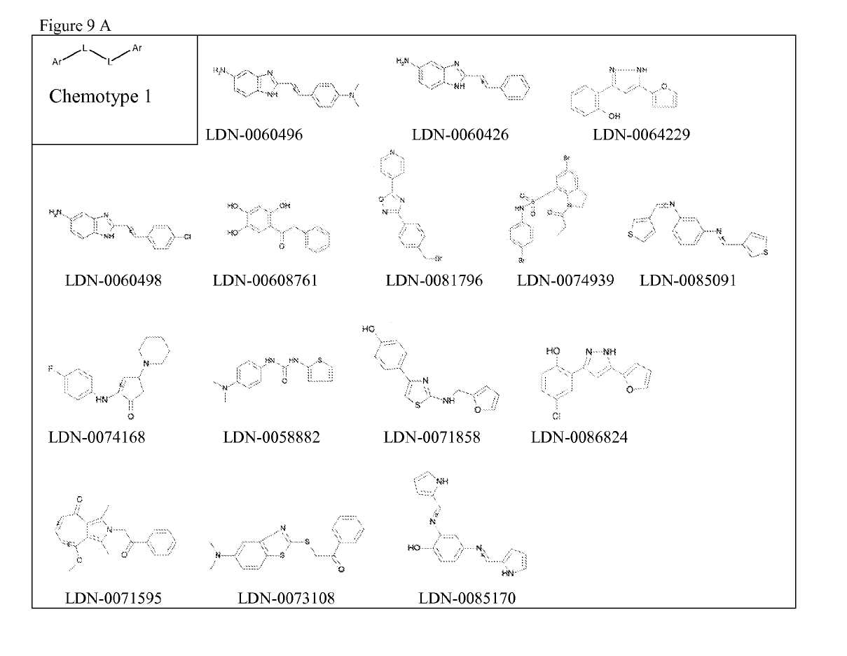

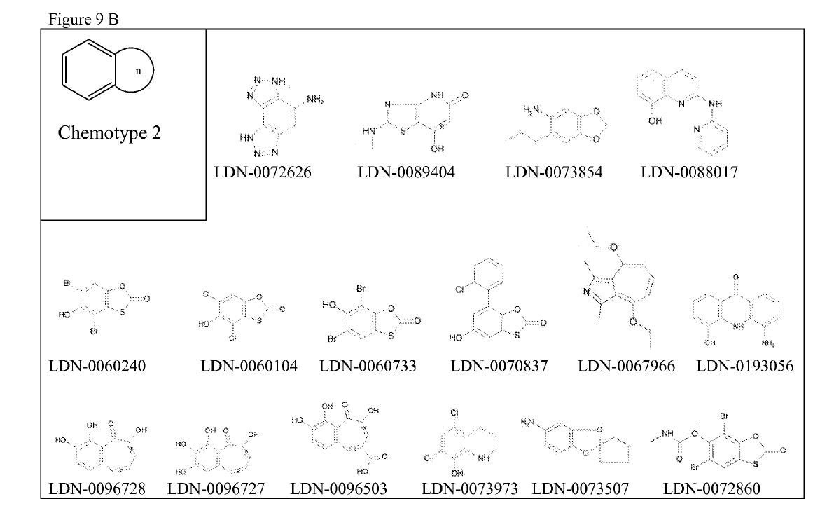

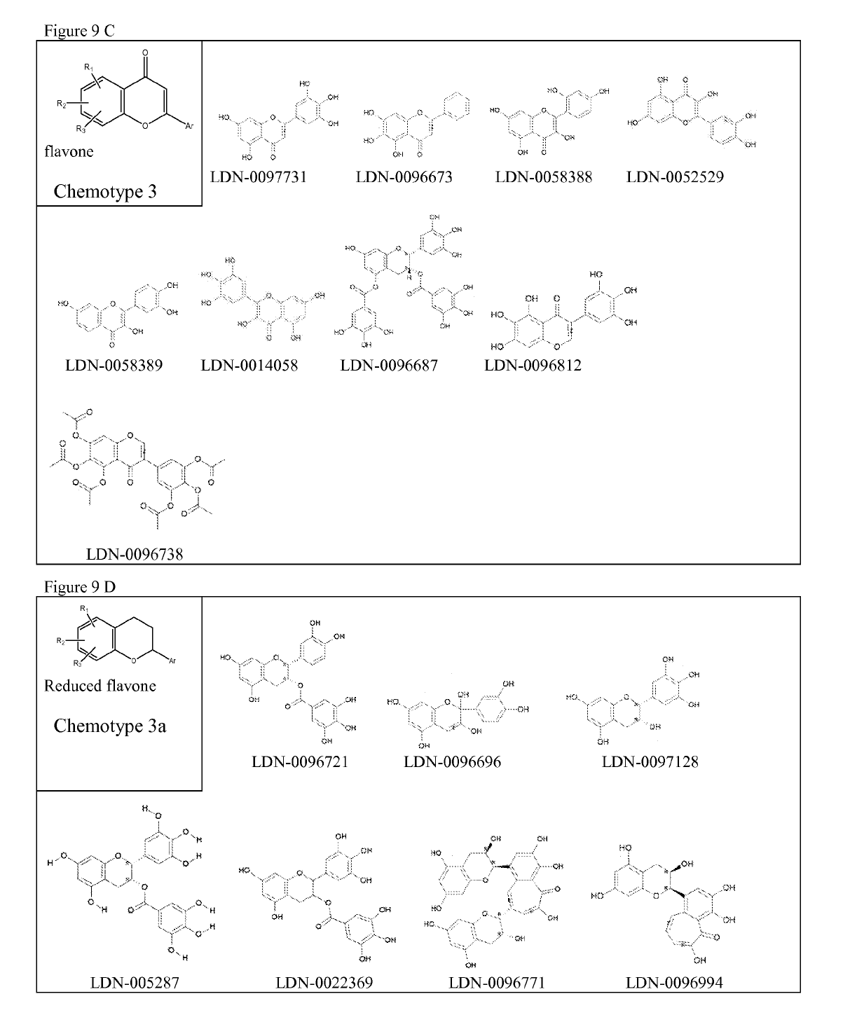

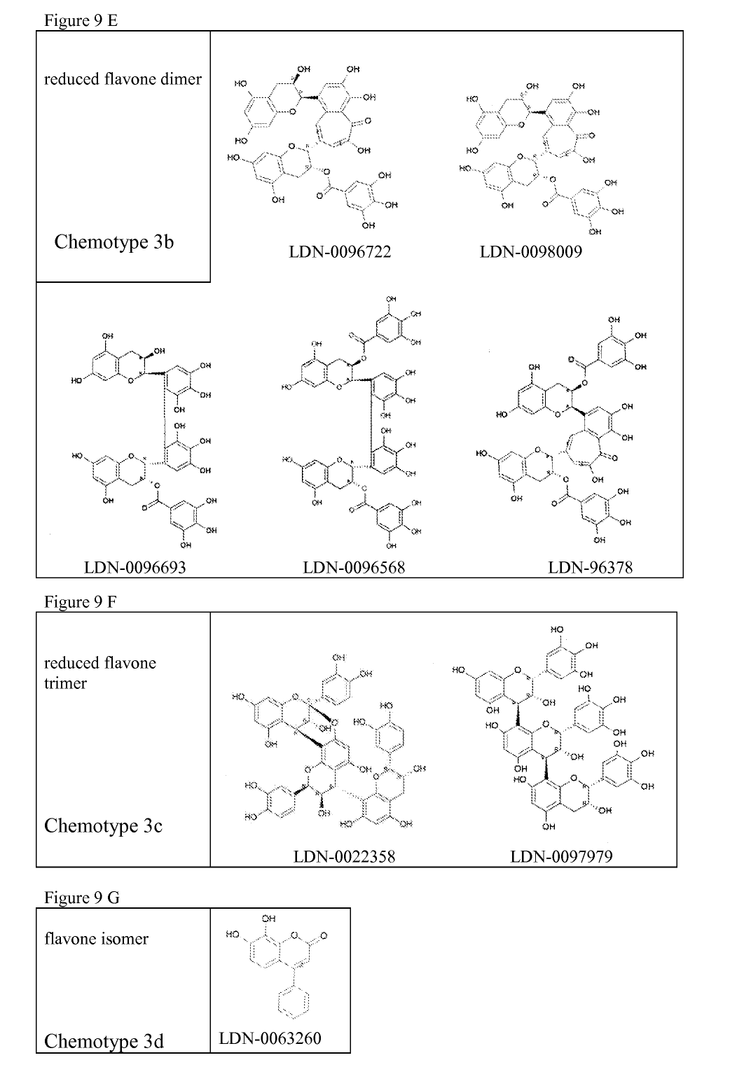

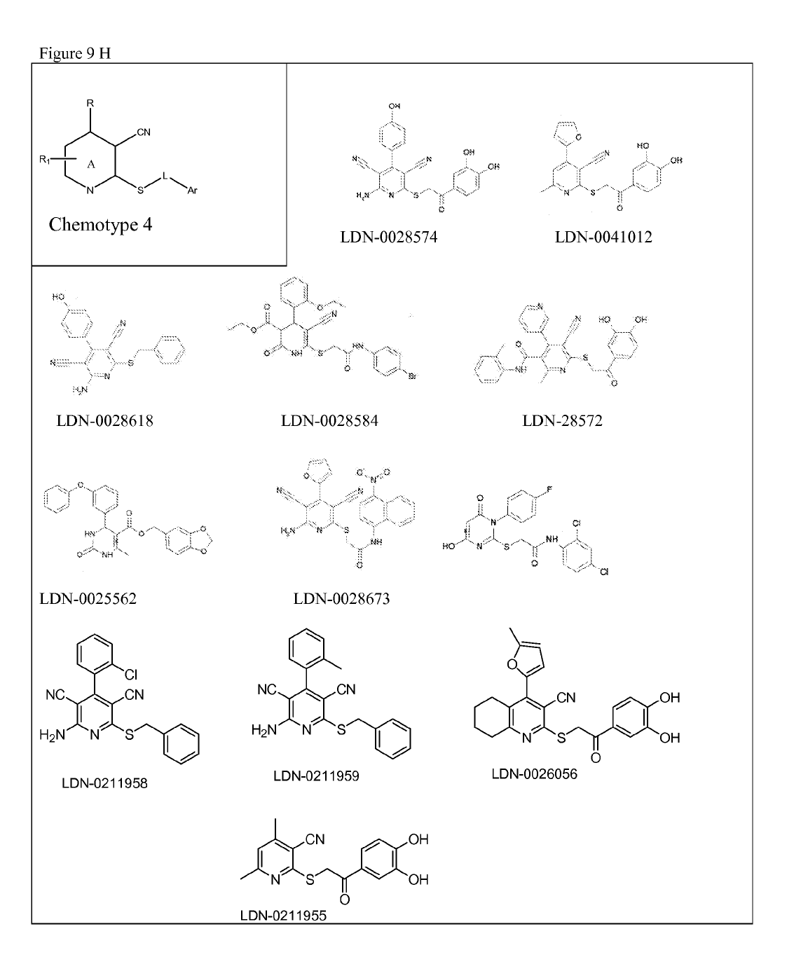

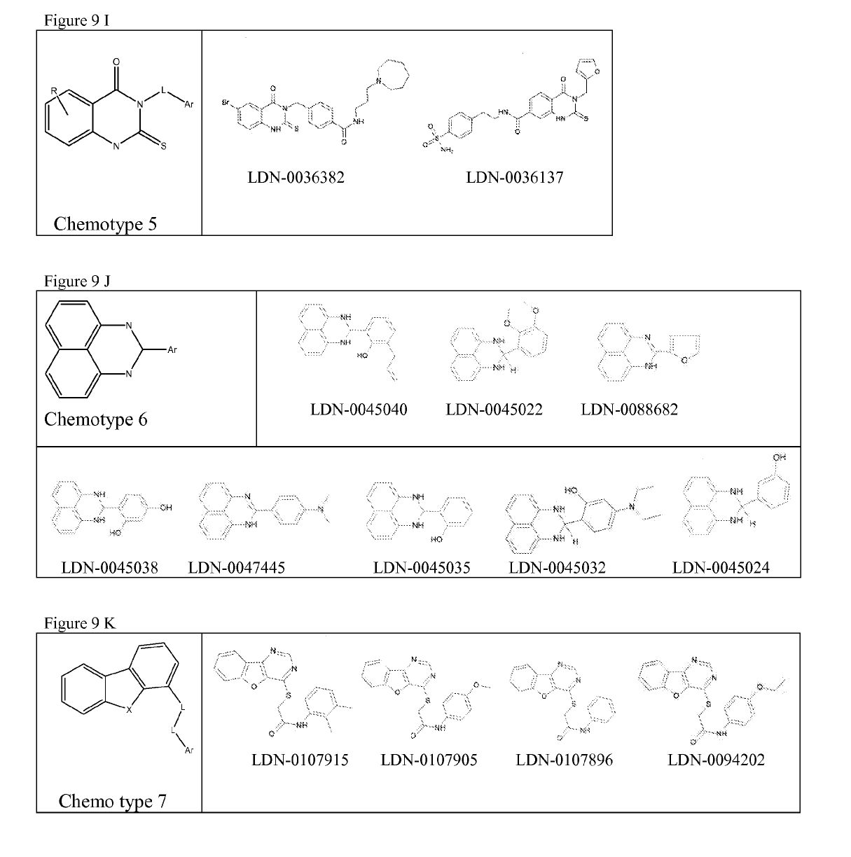

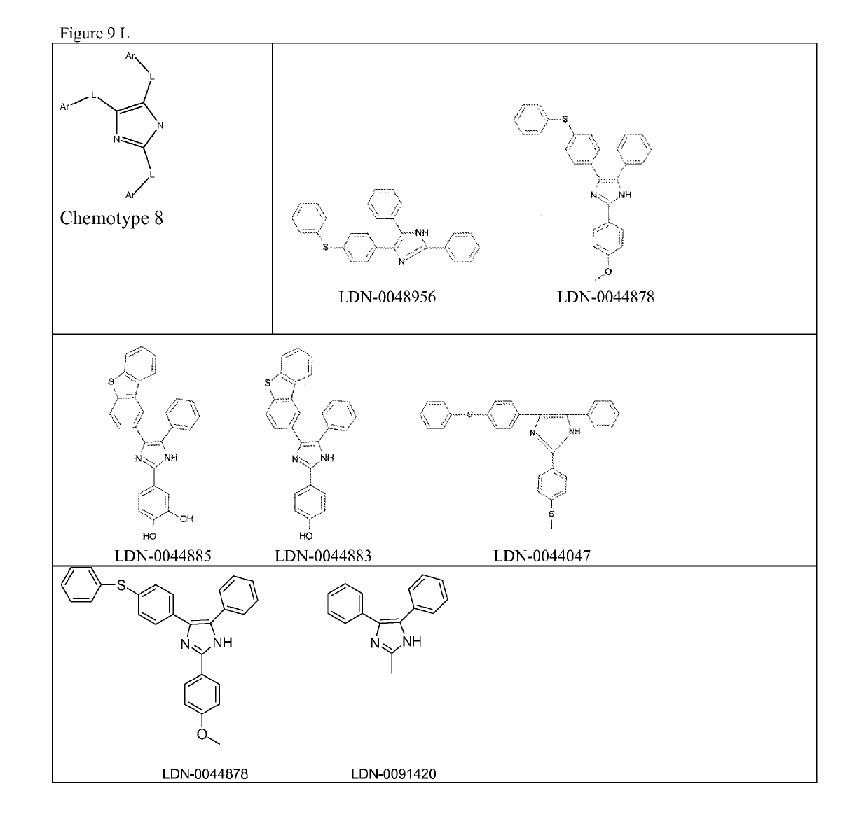

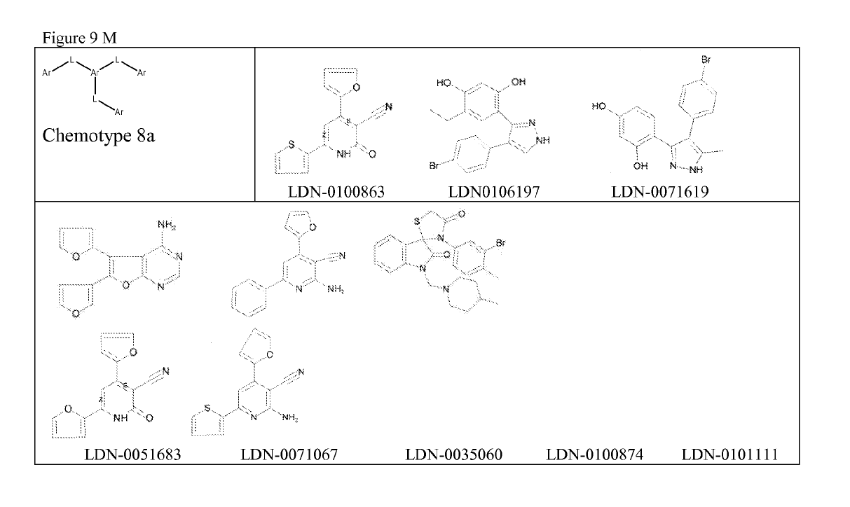

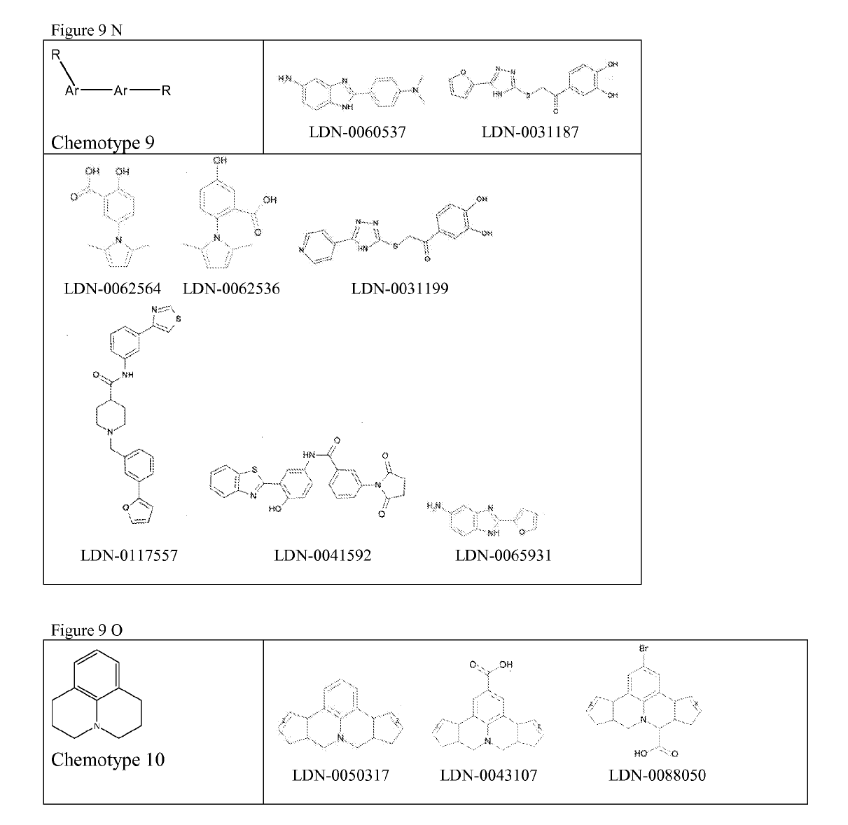

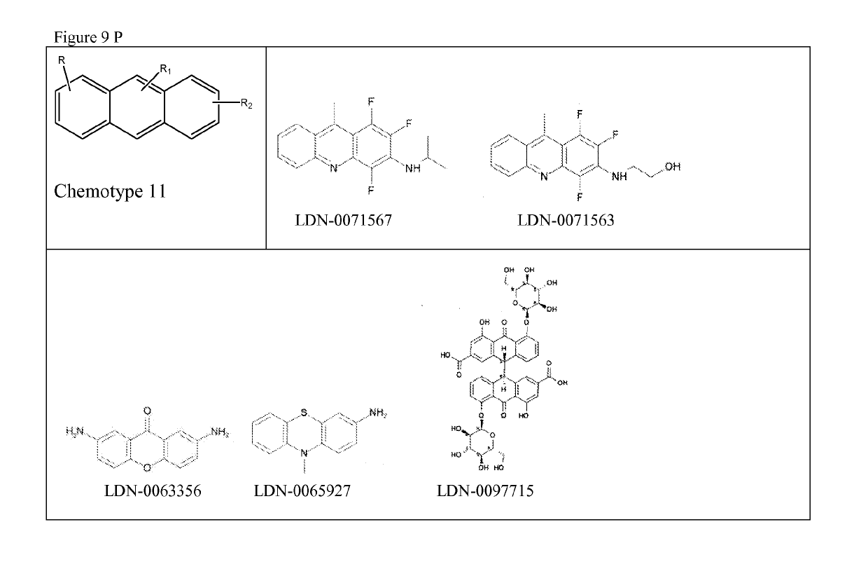

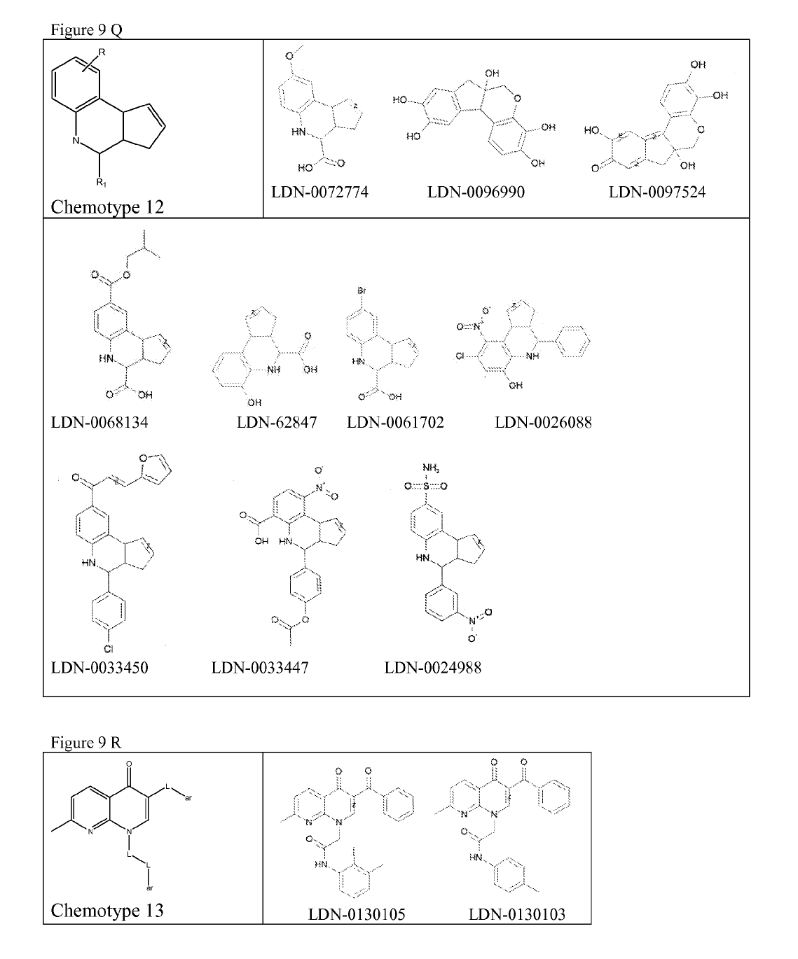

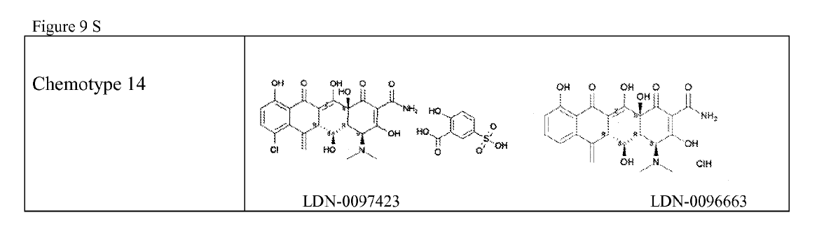

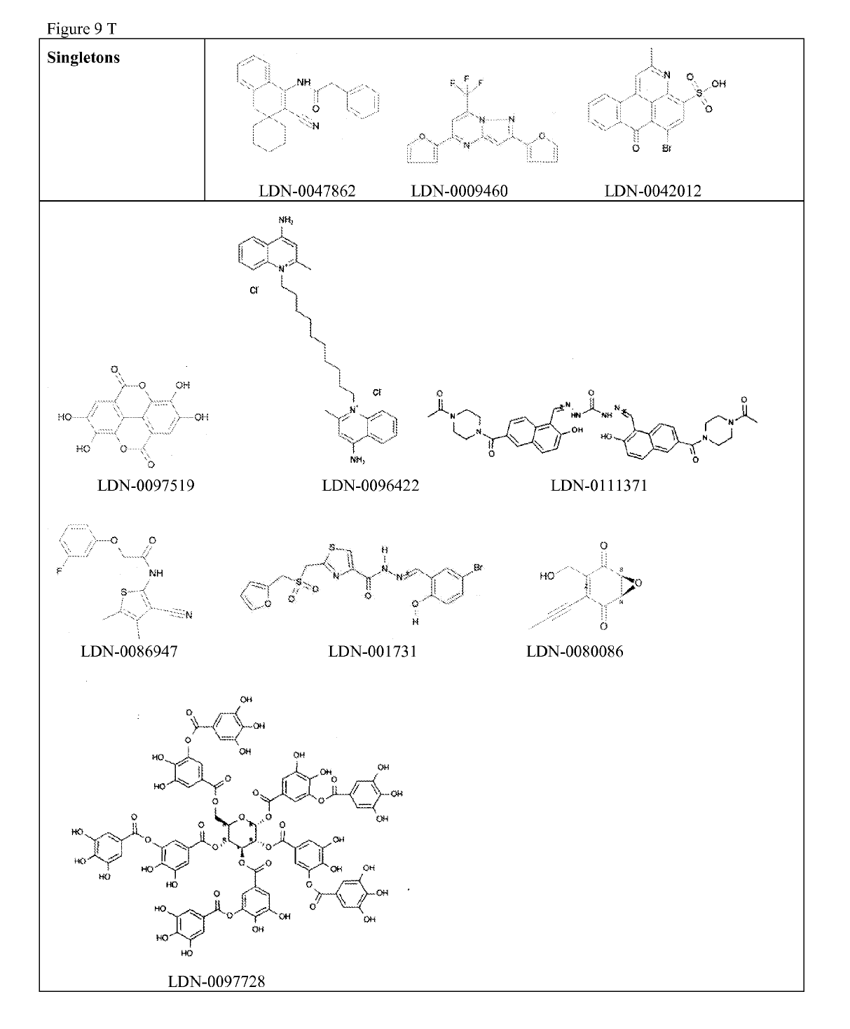

Analysis of the 130 structures included in the PAK3 data set revealed chemotypes shown in FIGS. 9A-T. In all, 14 chemotypes and 10 singletons were discovered. Most of the chemotypes are distinct, although there some overlap exists in some of the groupings. One of the chemotypes which is present in several subtypes is based on the flavone or isoflavone ring system. Generic chemotype structures are represented, as well as, specific cores which exist within the generic structures.

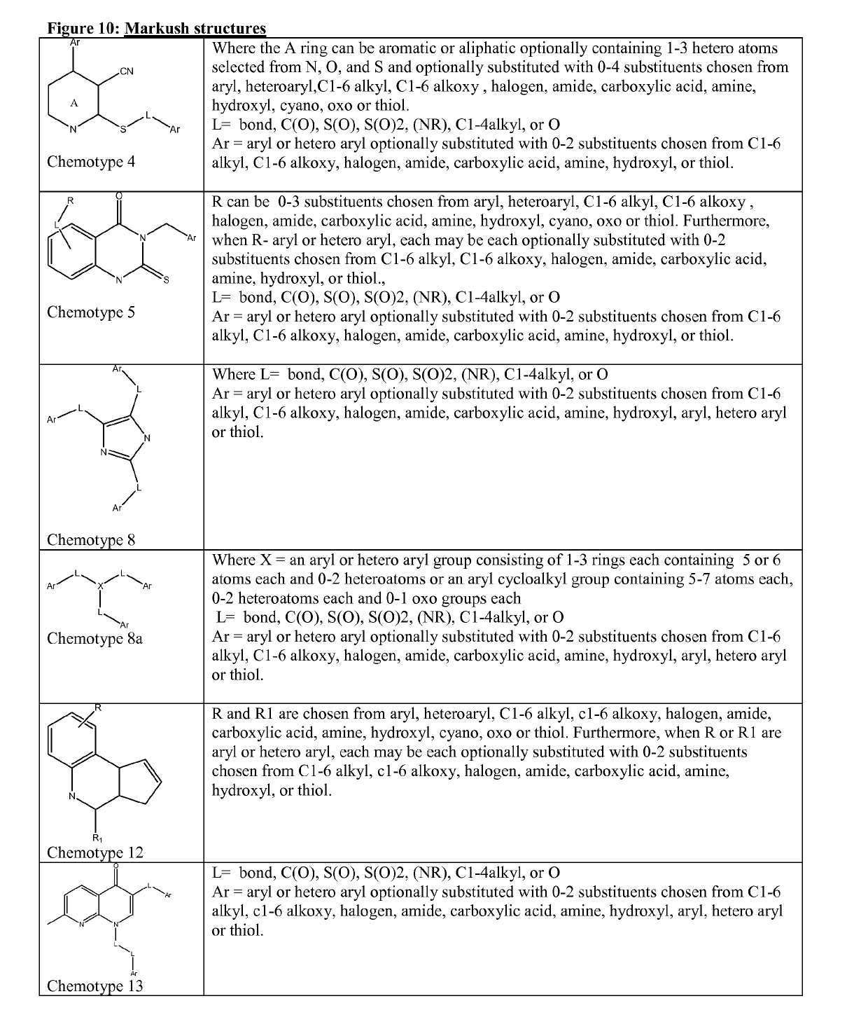

Chemotypes 1 and 2 are simple aromatic compounds. Chemotypes 3 are flavones. Chemotypes 6, 7, and 11 are flat poly aromatic compounds. Chemotypes 4 and 5 have several points of diversity and a linker, which can be varied to increase diversity. Chemotypes 8 and 8a have three aryl groups and three linkers, which can be varied independently to produce a large amount of variability. Chemotypes 12 and 13 also offer several points of diversity and linkers. Methods for synthesis of these compound is known in the art. FIG. 10 shows general structures for derivatives or analogs of PAK3 inhibitory compounds, grouped by chemotype.

Compounds were tested in a cell-based assay (HeLa cells). The results are summarized in the table below. IC50 and EC50 are expressed in micromolar units.

TABLE
Cell response to PAK3 inhibitory compounds

| | | | | | EC50 | EC50 | EC50 |
|---|---|---|---|---|---|---|---|
| cpd | LDN # | MW | IC50 | SD | 75 h | 100 h | 150 h |
| 1 | LDN-0028618 | 358.42 | 0.30 | ±0.18 | 13.3 | 16.6 | 18.1 |
| 2 | LDN-0211958 | 376.86 | 0.33 | ±0.16 | 11.2 | 11.6 | 10.6 |
| 3 | LDN-0211959 | 356.45 | 0.28 | ±0.3 | 11.1 | 12.7 | 10.4 |
| 4 | LDN-0041012 | 366.39 | 0.61 | ±0.36 | 4.6 | 4.6 | 3.4 |
| 5 | LDN-0026056 | 420.48 | 0.69 | ±0.35 | 13.9 | 16.9 | 18.0 |
| 6 | LDN-0211955 | 314.36 | 3.53 | ±3.5 | 17.6 | 34.0 | 23.3 |
| 7 | LDN-0044878 | 434.55 | 0.03 | ±0.01 | 10.7 | 15.5 | 11.1 |
| 8 | LDN-0091420 | 234.30 | 10.13 | ±7.5 | 38.2 | 40.7 | 42.5 |

### Small Molecule Inhibitors of SGK2

A medicinal chemistry evaluation of the SGK2 inhibitory compounds identified from the screen was also carried out. Clustera and chemotypes in the structures were identified. In addition, generic Markush structures, as well as, specific analogs are described for each chemotype.

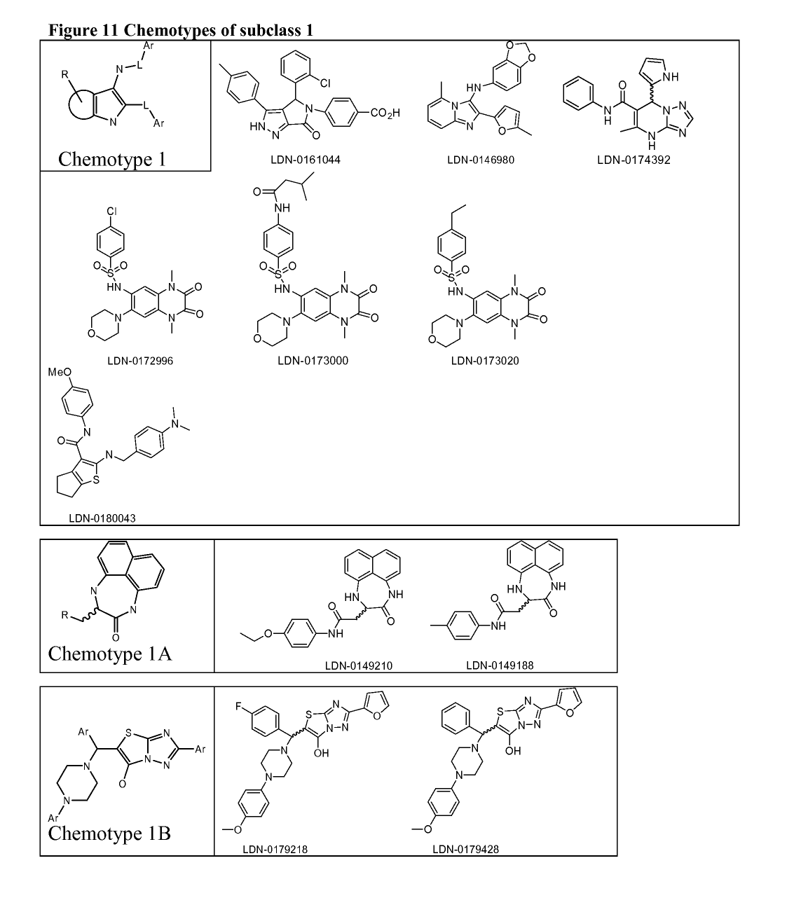

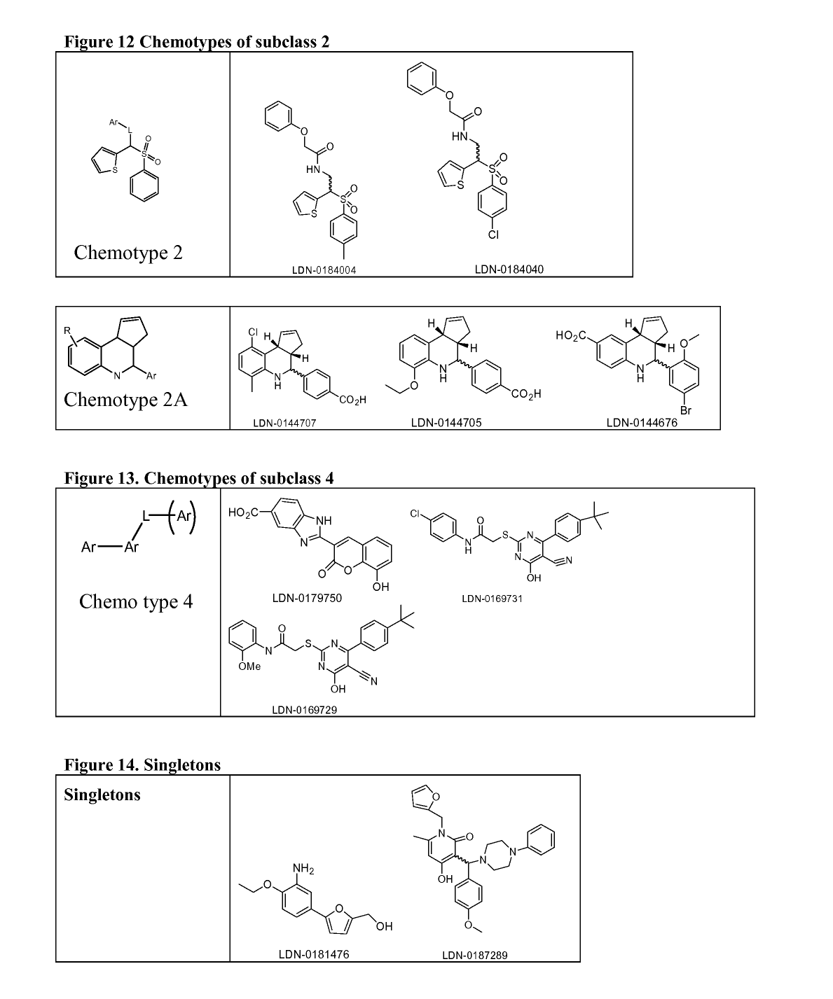

Upon analysis of the 22 structures included in the SKG2 data set, several general chemotypes emerged (FIGS. 11-14). Some of the chemotypes show structural overlap and as such, the overlapping chemotypes are represented as subsets of the parent chemotype. Generic chemotype structures are represented, as well as, specific cores which exist within the generic. FIG. 15 shows general structures for derivatives or analogs of SGK2 inhibitory compounds, grouped by chemotype.

Several compounds segregate into Chemotype 1. Chemotype 1 is characterized by a fused ring and the two pendant aryl groups. Chemotype 2 is constructed of an aryl alkyl sulfone moiety.

Characterization of SGK2 inhibitory compounds (inhibition of kinase activity) is summarized in the table below.

| | % Inhibition at |
|---|---|
| Compound ID | 10 μM |
| LDN-0009760 | 80 |
| LDN-0014058 | 81 |
| LDN-0017313 | 74 |
| LDN-0022358 | 93 |
| LDN-0022369 | 92 |
| LDN-0024988 | 82 |
| LDN-0025562 | 60 |
| LDN-0026088 | 70 |
| LDN-0028572 | 87 |
| LDN-0028574 | 91 |
| LDN-0028584 | 68 |
| LDN-0028618 | 87 |
| LDN-0028673 | 71 |
| LDN-0031187 | 67 |
| LDN-0031199 | 68 |
| LDN-0033447 | 65 |
| LDN-0033450 | 94 |
| LDN-0035060 | 64 |
| LDN-0036137 | 74 |
| LDN-0036382 | 67 |
| LDN-0041012 | 95 |
| LDN-0041592 | 74 |
| LDN-0042012 | 87 |
| LDN-0043107 | 73 |
| LDN-0044047 | 65 |
| LDN-0044878 | 79 |
| LDN-0044883 | 64 |
| LDN-0044885 | 70 |
| LDN-0045022 | 73 |
| LDN-0045024 | 90 |
| LDN-0045032 | 68 |
| LDN-0045035 | 89 |
| LDN-0045038 | 91 |
| LDN-0045040 | 89 |
| LDN-0047445 | 79 |
| LDN-0047862 | 95 |
| LDN-0048956 | 68 |
| LDN-0050317 | 69 |
| LDN-0051683 | 85 |
| LDN-0052529 | 89 |
| LDN-0052877 | 93 |
| LDN-0058388 | 84 |
| LDN-0058389 | 76 |
| LDN-0058882 | 61 |
| LDN-0060104 | 96 |
| LDN-0060240 | 94 |
| LDN-0060426 | 71 |
| LDN-0060496 | 67 |
| LDN-0060498 | 68 |
| LDN-0060537 | 72 |
| LDN-0060733 | 93 |
| LDN-0060861 | 85 |
| LDN-0061702 | 92 |
| LDN-0062536 | 95 |
| LDN-0062564 | 90 |
| LDN-0062847 | 95 |
| LDN-0063260 | 98 |
| LDN-0063356 | 50 |
| LDN-0064229 | 85 |
| LDN-0065927 | 52 |
| LDN-0065931 | 78 |
| LDN-0067966 | 77 |
| LDN-0068134 | 84 |
| LDN-0070837 | 68 |
| LDN-0071067 | 80 |
| LDN-0071492 | 75 |
| LDN-0071563 | 88 |
| LDN-0071567 | 88 |
| LDN-0071595 | 64 |
| LDN-0071619 | 75 |
| LDN-0071858 | 65 |
| LDN-0072626 | 72 |
| LDN-0072774 | 76 |
| LDN-0072860 | 72 |
| LDN-0073108 | 70 |
| LDN-0073507 | 74 |
| LDN-0073854 | 67 |
| LDN-0073973 | 67 |
| LDN-0074168 | 73 |
| LDN-0074939 | 67 |
| LDN-0080086 | 71 |
| LDN-0081796 | 86 |
| LDN-0085091 | 78 |
| LDN-0085170 | 62 |
| LDN-0086824 | 68 |
| LDN-0086947 | 67 |
| LDN-0088017 | 84 |
| LDN-0088050 | 67 |
| LDN-0088682 | 80 |
| LDN-0089404 | 86 |
| LDN-0094202 | 78 |
| LDN-0096378 | 87 |
| LDN-0096422 | 79 |
| LDN-0096503 | 73 |
| LDN-0096568 | 89 |
| LDN-0096663 | 70 |
| LDN-0096673 | 67 |
| LDN-0096693 | 89 |
| LDN-0096696 | 84 |
| LDN-0096697 | 91 |
| LDN-0096721 | 93 |
| LDN-0096722 | 90 |
| LDN-0096727 | 94 |
| LDN-0096728 | 87 |
| LDN-0096738 | 96 |
| LDN-0096771 | 89 |
| LDN-0096812 | 97 |
| LDN-0096990 | 91 |
| LDN-0096994 | 92 |
| LDN-0097128 | 68 |
| LDN-0097423 | 76 |
| LDN-0097519 | 85 |
| LDN-0097524 | 93 |
| LDN-0097715 | 94 |
| LDN-0097728 | 85 |
| LDN-0097731 | 87 |
| LDN-0097979 | 85 |
| LDN-0098009 | 91 |
| LDN-0100863 | 78 |
| LDN-0100874 | 81 |
| LDN-0101111 | 62 |
| LDN-0106197 | 67 |
| LDN-0107896 | 73 |
| LDN-0107905 | 66 |
| LDN-0107915 | 67 |
| LDN-0111371 | 91 |
| LDN-0117557 | 60 |
| LDN-0130103 | 62 |
| LDN-0130105 | 70 |
| LDN-0193056 | 92 |

### Methods of Treatment

Therapeutic methods are carried out by administering pharmaceutical formulations comprising kinase inhibitory compounds. The compounds are administered to subjects (e.g., human patients, companion animals such as dogs and cats, livestock such as cattle, sheep, goats, horses) that have been determined to be suffering from or at risk of developing a p53-deficient tumor. A reduction (deficiency) in p53 expression or a loss of p53 expression in a cell or tissue is determined by detecting the p53 gene product (e.g., using a p53-specific monoclonal antibody) or by measuring p53 nucleic acid (e.g., transcripts) in a cell or tissue sample such as a tumor biopsy specimen.

Routes of administration, include, but are not limited to, oral, rectal, topical, intravenous, parenteral (including, but not limited to, intramuscular, intravenous), ocular (ophthalmic), transdermal, inhalative (including, but not limited to, pulmonary, aerosol inhalation), nasal, sublingual, subcutaneous or intraarticular delivery. Although the most suitable route in any given case will depend on the nature and severity of the conditions being treated and on the nature of the active ingredient. The compounds are formulated in unit dosage form and prepared using methods well-known in the art of pharmacy.

A pharmaceutical composition or medicament containing the inhibitor or a mixture of inhibitors is administered to a patient at a therapeutically effective dose to prevent, treat, or control cancer. The pharmaceutical composition or medicament is administered to a patient in an amount sufficient to elicit an effective therapeutic response in the patient. An effective therapeutic response is a response that at least partially arrests or slows the symptoms or complications of the disease. An amount adequate to accomplish this is defined as “therapeutically effective dose.”

The dosage of active small molecule compound administered is dependent on the species of warm-blooded animal (mammal), the body weight, age, individual condition, surface area of the area to be treated and on the form of administration. The size of the dose also is determined by the existence, nature, and extent of any adverse effects that accompany the administration of a particular small molecule compound in a particular subject. A unit dosage for oral administration to a mammal of about 50 to 70 kg may contain between about 5 and 500 mg of the active ingredient. Typically, a dosage of the active small molecule compound of the present invention, is a dosage that is sufficient to achieve a therapeutic effect, e.g., reduced proliferation of tumor cells, death of tumor cells, and/or reduction in tumor burden or tumor mass.

Optimal dosing schedules can be calculated from measurements of small molecule compound accumulation in the body of a subject. In general, dosage is from 1 ng to 1,000 mg per kg of body weight and may be given once or more daily, weekly, monthly, or yearly. Persons of ordinary skill in the art can readily determine optimum dosages, dosing methodologies and repetition rates. For example, a pharmaceutical composition or medicament comprising a small molecule compound of the present invention is administered in a daily dose in the range from about 1 mg of small molecule compound per kg of subject weight (1 mg/kg) to about 1 g/kg for multiple days, e.g., the daily dose is a dose in the range of about 5 mg/kg to about 500 mg/kg, about 10 mg/kg to about 250 mg/kg, or about 25 mg/kg to about 150 mg/kg. The daily dose is administered once per day or divided into subdoses and administered in multiple doses, e.g., twice, three times, or four times per day.

To achieve the desired therapeutic effect, a small molecule compound is typically administered for multiple days at the therapeutically effective daily dose. Thus, therapeutically effective administration of a small molecule compound to treat cancer in a subject often requires periodic (e.g., daily) administration that continues for a period ranging from three days to two weeks or longer. Typically, a small molecule compound will be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the small molecule compound is not administered daily, so long as the administration is repeated frequently enough to maintain a therapeutically effective concentration of the small molecule compound in the subject. For example, one can administer the small molecule compound every other day, every third day, or, if higher dose ranges are employed and tolerated by the subject, once a week.

Optimum dosages, toxicity, and therapeutic efficacy of such small molecule compounds may vary depending on the relative potency of individual small molecule compounds and are determined by standard pharmaceutical procedures in cell cultures or experimental animals, for example, by determining the LD50 (the dose lethal to 50% of the population) and the ED50 (the dose therapeutically effective in 50% of the population). The dose ratio between toxic and therapeutic effects is the therapeutic index and can be expressed as the ratio, LD50/ED50. Compounds that exhibit large therapeutic indices are preferred. While compounds that exhibit toxic side effects can be used, care should be taken to design a delivery system that targets such compounds to the site of affected tissue to minimize potential damage to normal cells and, thereby, reduce side effects. The SGK2 and PAK3 inhibitory compounds described herein are characterized by minimal adverse side effects, because they preferentially affect p53-deficient cells, e.g., tumor cells, while sparing normal non-tumor cells.

The therapeutically effective dose is estimated initially from cell culture assays. A dose can be formulated in animal models to achieve a circulating plasma concentration range that includes the 1050 (the concentration of the test compound that achieves a half-maximal inhibition of symptoms) as determined in cell culture. Such information is then used to more accurately determine useful doses in humans. Levels in plasma are measured, for example, by high performance liquid chromatography (HPLC). In general, the dose equivalent of a small molecule compound is from about 1 ng/kg to 100 mg/kg for a typical subject.

### Additional Chemical Terms and Definitions

As used herein, the term “alkyl” includes saturated aliphatic groups, including straight-chain alkyl groups (e.g., methyl, ethyl, propyl, butyl, pentyl, hexyl) and branched-chain alkyl groups (e.g., isopropyl, tert-butyl, isobutyl. In certain embodiments, a straight chain or branched chain alkyl has six or fewer carbon atoms in its backbone (e.g., C₁-C₆ for straight chain, C₃-C₆ for branched chain), and in other embodiments four or fewer carbon atoms. Lower alkyl groups include from 1-6 carbon atoms, thus the term “lower alkyl” includes alkyl groups containing 1, 2, 3, 4, 5, or 6 carbon atoms.

The term “alkoxy” or “alkoxyl” includes substituted and unsubstituted alkyl groups covalently linked to an oxygen atom. Examples of alkoxy groups (or alkoxyl radicals) include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, carboxylate, alkoxyl, cyano, amino (including —NH₂, alkylamino, dialkylamino, arylamino, diarylamino, and alkylarylamino), nitro, trifluoromethyl, cyano, azido, heterocyclyl, or an aromatic or heteroaromatic moiety. Examples of halogen substituted alkoxy groups include, but are not limited to, fluoromethoxy, difluoromethoxy, trifluoromethoxy, chloromethoxy, dichloromethoxy, and trichloromethoxy. Lower alkoxy groups include from 1-6 carbon atoms, thus the term “lower alkoxy” includes alkyl groups containing 1, 2, 3, 4, 5, or 6 carbon atoms.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

In the present specification, the structural formula of the compound represents a certain isomer for convenience in some cases, but the present invention includes all isomers such as geometrical isomer, optical isomer based on an asymmetrical carbon, stereoisomer, tautomer and the like which occur structurally and an isomer mixture and is not limited to the description of the formula for convenience, and may be any one of isomer or a mixture. Therefore, an asymmetrical carbon atom may be present in the molecule and an optically active compound and a racemic compound may be present in the present compound, but the present invention is not limited to them and includes any one. In addition, a crystal polymorphism may be present but is not limiting, but any crystal form may be single or a crystal form mixture, or an anhydride or hydrate. Further, so-called metabolite which is produced by degradation of the present compound in vivo is included in the scope of the present invention.

It will be noted that the structure of some of the compounds of the invention include asymmetric (chiral) carbon atoms. It is to be understood accordingly that the isomers arising from such asymmetry are included within the scope of the invention, unless indicated otherwise. Such isomers can be obtained in substantially pure form by classical separation techniques and by stereochemically controlled synthesis. The compounds of this invention may exist in stereoisomeric form, therefore can be produced as individual stereoisomers or as mixtures.

“Isomerism” means compounds that have identical molecular formulae but that differ in the nature or the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images are termed “enantiomers”, or sometimes optical isomers. A carbon atom bonded to four nonidentical substituents is termed a “chiral center”.

“Chiral isomer” means a compound with at least one chiral center. It has two enantiomeric forms of opposite chirality and may exist either as an individual enantiomer or as a mixture of enantiomers. A mixture containing equal amounts of individual enantiomeric forms of opposite chirality is termed a “racemic mixture”. A compound that has more than one chiral center has 2ⁿ⁻¹ enantiomeric pairs, where n is the number of chiral centers. Compounds with more than one chiral center may exist as either an individual diastereomer or as a mixture of diastereomers, termed a “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al, Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J., Chem. Educ. 1964, 41, 116).

“Geometric Isomers” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Further, the structures and other compounds discussed in this application include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

The terms “crystal polymorphs” or “polymorphs” or “crystal forms” means crystal structures in which a compound (or salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Additionally, the compounds of the present invention, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvates” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H2O, such combination being able to form one or more hydrate.

“Tautomers” refers to compounds whose structures differ markedly in arrangement of atoms, but which exist in easy and rapid equilibrium. It is to be understood that compounds of Formula I may be depicted as different tautomers. It should also be understood that when compounds have tautomeric forms, all tautomeric forms are intended to be within the scope of the invention, and the naming of the compounds does not exclude any tautomer form. Some compounds of the present invention can exist in a tautomeric form which are also intended to be encompassed within the scope of the present invention.

The compounds, salts and prodrugs of the present invention can exist in several tautomeric forms, including the enol and imine form, and the keto and enamine form and geometric isomers and mixtures thereof. All such tautomeric forms are included within the scope of the present invention. Tautomers exist as mixtures of a tautomeric set in solution. In solid form, usually one tautomer predominates. Even though one tautomer may be described, the present invention includes all tautomers of the present compounds

A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism, is exhibited by glucose. It arises as a result of the aldehyde group (—CHO) in a sugar chain molecule reacting with one of the hydroxy groups (—OH) in the same molecule to give it a cyclic (ring-shaped) form.

Tautomerizations are catalyzed by: Base: 1. deprotonation; 2. formation of a delocalized anion (e.g. an enolate); 3. protonation at a different position of the anion; Acid: 1. protonation; 2. formation of a delocalized cation; 3. deprotonation at a different position adjacent to the cation.

Common tautomeric pairs are: ketone-enol, amide-nitrile, lactam-lactim, amide-imidic acid tautomerism in heterocyclic rings (e.g. in the nucleobases guanine, thymine, and cytosine), amine-enamine and enamine-enamine. Examples include:

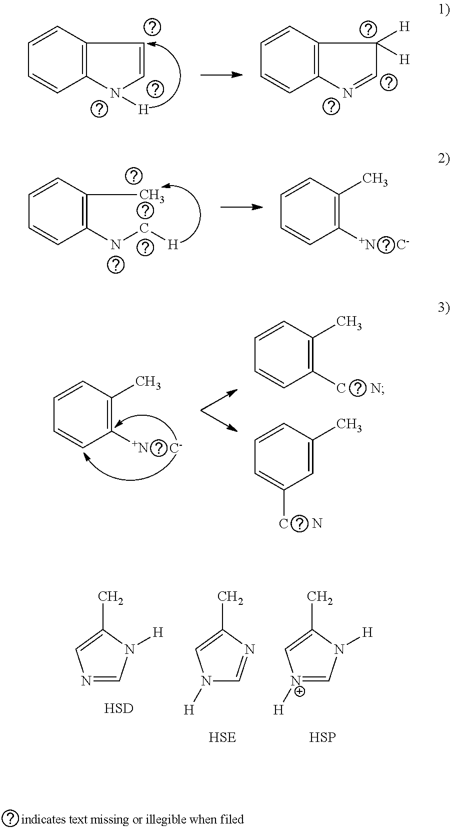

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative”, refers to compounds that have a common core structure, and are substituted with various groups as described herein. For example, all of the compounds represented by formula I are indole derivatives, and have formula I as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include acyl sulfonimides, tetrazoles, sulfonates, and phosphonates. See, e.g., Patani and LaVoie, Chem. Rev. 96, 3147-3176 (1996).

A “pharmaceutical composition” is a formulation containing the disclosed compounds in a form suitable for administration to a subject.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient. The compounds of the invention are capable of further forming salts. All of these forms are also contemplated within the scope of the claimed invention. For example, the salt can be an acid addition salt. One example of an acid addition salt is a hydrochloride salt. Another example is a hydrobromide salt.

“Pharmaceutically acceptable salt” of a compound means a salt that is pharmaceutically acceptable and that possesses the desired pharmacological activity of the parent compound.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The invention also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The pharmaceutically acceptable salts of the present invention can be synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed. (Mack Publishing Company, 1990). For example, salts can include, but are not limited to, the hydrochloride and acetate salts of the aliphatic amine-containing, hydroxylamine-containing, and imine-containing compounds of the present invention.

The compounds of the present invention can also be prepared as prodrugs, for example pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.) the compounds of the present invention can be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a subject. Prodrugs the present invention are prepared by modifying functional groups present in the compound in such a way that the modifications are cleaved, either in routine manipulation or in vivo, to the parent compound. Prodrugs include compounds of the present invention wherein a hydroxy, amino, sulfhydryl, carboxy, or carbonyl group is bonded to any group that, may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates, and benzoate derivatives) and carbamates (e.g., N,N-dimethylaminocarbonyl) of hydroxy functional groups, esters groups (e.g. ethyl esters, morpholinoethanol esters) of carboxyl functional groups, N-acyl derivatives (e.g. N-acetyl) N-Mannich bases, Schiff bases and enaminones of amino functional groups, oximes, acetals, ketals and enol esters of ketone and aldehyde functional groups in compounds of formula I, and the like, See Bundegaard, H. “Design of Prodrugs” p1-92, Elesevier, N.Y.-Oxford (1985).

“Protecting group” refers to a grouping of atoms that when attached to a reactive group in a molecule masks, reduces or prevents that reactivity. Examples of protecting groups can be found in Green and Wuts, Protective Groups in Organic Chemistry, (Wiley, 2^{nd} ed. 1991); Harrison and Harrison et al., Compendium of Synthetic Organic Methods, Vols. 1-8 (John Wiley and Sons, 1971-1996); and Kocienski, Protecting Groups, (Verlag, 3^{rd} ed. 2003).

For example, representative hydroxy protecting groups include those where the hydroxy group is either acylated or alkylated such as benzyl, and trityl ethers as well as alkyl ethers, tetrahydropyranyl ethers, trialkylsilyl ethers and allyl ethers.

Stable compound” and “stable structure” are meant to indicate a compound that is sufficiently robust to survive isolation to a useful degree of purity from a reaction mixture, and formulation into an efficacious therapeutic agent.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. (canceled)

2. The method of claim 5, wherein said inhibitor comprises the general structure of PAK3 inhibitor Chemotype 4, and wherein said inhibitor is selected from the group consisting of LDN-0211958, LDN-0211959, LDN-0026056, LDN-0211955, LDN-0041012, and LDN-0028618.

3. (canceled)

4. The method of claim 5, wherein said inhibitor comprises the general structure of PAK3 inhibitor Chemotype 8, and wherein said inhibitor is LDN-0044878 or LDN-0091420.

5. A method of inhibiting proliferation of or killing a p53-deficient cell, comprising contacting said cell with a composition comprising an inhibitor of PAK3, wherein said inhibitor comprises a general structure selected from PAK3 inhibitor Chemotypes 1, 2, 3, 3a, 3b, 3c, 3d, 4, 5, 6, 7, 8, 8a, 9, 10, 11, 12, 13, 14.

6. A method of inhibiting proliferation of or killing a p53-deficient cell, comprising contacting said cell with a composition comprising an inhibitor of PAK3, wherein said inhibitor comprises LDN-0047862, LDN-0009460, LDN-0042112, LDN-0097519, LDN-0096422, LDN-0111371, LDN-0086947, LDN-001731, LDN-0080086, or LDN-0097728.

7. (canceled)

8. The method of claim 13, wherein said inhibitor comprises the general structure of SGK2 inhibitor Chemotype 1A, and wherein said compound is LDN-0149188.

9. (canceled)

10. The method of claim 13, wherein said inhibitor comprises the general structure of SGK2 inhibitor Chemotype 2A, and wherein said inhibitor is selected from the group consisting of LDN-0144705 and LDN-0144676.

11. (canceled)

12. The method of claim 13, wherein said inhibitor comprises the general structure of SGK2 inhibitor Chemotype 4, and, wherein said inhibitor comprises LDN-0169731.

13. A method of inhibiting proliferation of or killing a p53-deficient cell, comprising contacting said cell with a composition comprising an inhibitor of SGK2, wherein said inhibitor comprises a general structure selected from SGK2 inhibitor Chemotypes 1, 1A, 1B, 2, 2A, and 4.

14. A method of inhibiting proliferation of or killing a p53-deficient cell, comprising contacting said cell with a composition comprising an inhibitor of SGK2, wherein said inhibitor comprises LDN-0181476 or LDN-0187289.

15. The method of claim 5, 6, 13 or 14, wherein said cell is a p53 deficient tumor cell.

16. The method of claim 5, 6, 13 or 14, wherein said cell is a human papilloma virus (HPV)-infected cell.

17. The method of claim 5, 6, 13 or 14, wherein said cell is a non-tumor cell expressing an HPV oncoprotein.

18. The method of claim 5, 6, 13 or 14, wherein said cell is a tumor cell or tumor cell line of a tissue type selected from the group consisting of breast, cervix, uterus, bladder, brain, lung, esophagus, liver, and prostate.

19. A method of identifying an anti-tumor agent for inhibition of p53 deficient tumor cells, comprising contacting tumor survival kinase with a candidate compound and determining whether said candidate compound inhibits enzymatic activity of said kinase, wherein a reduction in a level of said activity in the presence of said candidate compound compared to that in the absence of said candidate compound indicates that said candidate compound preferentially inhibits p53 deficient tumor cells.

20. A method of identifying an anti-tumor agent for inhibition of p53 deficient tumor cells, comprising contacting a cell dependent upon a tumor survival kinase with a candidate compound and determining whether said candidate compound inhibits survival or proliferation of said cell, wherein a reduction in a level of said survival or proliferation in the presence of said candidate compound compared to that in the absence of said candidate compound indicates that said candidate compound preferentially inhibits p53 deficient tumor cells.

21. The method of claim 19 or 20, wherein said tumor survival kinase is selected from the group consisting of a serum- and glucocorticoid-induced protein kinase (SGK), a p21-activated kinase (PAK), or a cyclin-dependent protein kinase (CDK).

22. The method of claim 21, wherein said SGK is SGK2, wherein said PAK is PAK3, and wherein said CDK is CDK7.

23. A method of identifying a tumor survival kinase, comprising synthetically inhibiting expression of a tumor-associated gene and expression of at least one candidate kinase gene, wherein a decrease in tumor cell survival in the presence of inhibition of both genes compared to the level of tumor cell survival in the presence of inhibition of solely said tumor-associated gene indicates that said candidate kinase gene is a tumor survival kinase.

24-26. (canceled)
